(19) United States
(12) Patent Application Publication
(10) Pub. No.: US 2005/0267126 A1
(43) Pub. Date: Dec. 1, 2005

(54) Compounds derived from aryl carbamates, preparation thereof and uses of same

(76) Inventors: Magali Maillet, Suresnes (FR); Laurent Saniere, Paris (FR); Eric Nicolai, Rueil Malmaison (FR); Dominique Potin, Epone (FR); Michel Langlois, Sceaux (FR)

Correspondence Address: NIXON & VANDERHYE, PC, 901 NORTH GLEBE ROAD, 11TH FLOOR, ARLINGTON, VA, 22203, US

(21) Appl. No.: 10/524,875

(22) PCT Filed: Aug. 22, 2003
(86) PCT No.: PCT/FR03/02564

§ 371 Date: Apr. 6, 2005

(30) Foreign Application Priority Data
Aug. 23, 2002 (FR) 02/10542

(51) Int. Cl. A61K 31/496; C07D 43/02

(52) U.S. Cl. 514/253.12; 544/360

(57) ABSTRACT

The invention concerns novel compounds, preparation and uses of same, particularly therapeutic. More particularly, the invention concerns compounds derived from arylcarbamates, preparation and uses of same, particularly in the field of human or animal health. The inventive compounds are preferably ligands of serotoninergic 5-HT4 receptors, and can therefore be used in the therapeutic or prophylactic treatment of any disorder involving a 5-HT4 receptor. The invention also relates to pharmaceutical compositions comprising such compounds, preparation and uses thereof, and treatment methods using said compounds.

The invention relates to novel compounds, the preparation thereof and uses of same, particularly therapeutic. More particularly, the invention concerns compounds derived from arylcarbamates, preparation and uses of same, particularly in the field of human or animal health. The inventive compounds are preferably 5-HT4 serotoninergic receptor ligands, and can therefore be used in the therapeutic or prophylactic treatment of any disorder involving a 5-HT4 receptor. The invention also relates to pharmaceutical compositions comprising said compounds, preparation and uses thereof, and treatment methods using said compounds.

A large number of serotonin-dependent processes have been identified to date, and many molecules acting at serotonin receptors are used in human therapeutics. More than a dozen serotonin receptors have been identified and one of the most recent is the 5-HT4 receptor (J. Bockaert et al., Trends Pharmacol. Sci., 13, 141, 1992). The invention relates to arylcarbamate derivatives represented by general formula (I) and the pharmacologically acceptable salts of same. Preferably these are compounds capable of interfering with serotonin-dependent processes, even more preferably they are ligands of 5-HT4 receptors, particularly of the human serotype. The invention thus provides methods of treatment or prophylaxis of any disorder involving a 5-HT4 receptor. The inventive compounds and compositions can prove to be useful for the prophylactic or therapeutic treatment of various pathologies such as:
different gastrointestinal disorders such as gastroesophageal reflux disease (GERD), irritable bowel syndrome, functional dyspepsia, gastroparesis, disorders related to gastrointestinal motility, nausea, and constipation,
cardiac disorders such as atrial fibrillation, arrhythmia and tachycardia,
urological disorders such as urine retention, urinary incontinence,
central nervous system disorders such as, in particular, anxiety, schizophrenia, obsessional behaviors (like bulimia or anorexia), depression, memory impairment and dementia,
migraine and pain.

A first object of the invention is based on a compound represented by general formula (I): 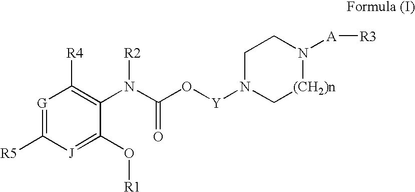
in which:
R₁ represents a lower alkyl, aryl, halogenoalkyl or lower arylalkyl group,
R₂ represents the hydrogen atom or a lower alkyl group,
A represents an aryl or heterocycle group, said group possibly being substituted by a substituent other than R₃,
R₃ represents a group selected from among the following: 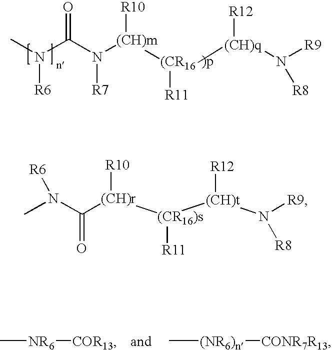
the R₇-R₁₂ groups, which are the same or different, represent the hydrogen atom, an aryl group, a heteroaryl group, a heterocycle group, an arylalkyl group, a heteroarylalkyl group, a heterocycloalkyl group, a lower alkyl group, a cycloalkyl group, an alkoxyalkyl group, an alkylaminoalkyl group, an alkyl-COOR₁₇ group,
the R₇-R₁₂ groups, taken two by two, can additionally form, together with the linear chain which supports them, at least one ring saturated or not, such as in particular cycloalkyl, cycloalkylene, heterocycle,
the R₁₀-R₁₂ groups can also represent a —COOR₁₇ group,
R₁₃ represents a lower alkyl group, a cycloalkyl group, an aryl group, a heterocycle, an arylalkyl group, a heteroarylalkyl group, a heterocycloalkyl group, a cycloalkylcarboxy group, an alkyl-COOR₁₇ group, an alkoxyalkyl group, an imidazopyridinylalkyl group, a trifluoroalkyl group or a heteroarylthioalkyl group, it being understood that R₁₃ cannot represent the methyl or the ethyl group, in the case where A represents a phenyl, R₂ represents the hydrogen atom, G and J represent the CH group, R₃ represents NR₆COR₁₃ or —(NR₆)ₙ, —CONR₇R₁₃ with R₆ and/or R₇ representing the hydrogen atom,
n is 1 or 2; n′ is 0 or 1, m, p, q, r, s and t are integers comprised between 0 and 2 inclusive, r, s and t not simultaneously being 0,
Y represents a linear or branched alkylene chain, having from 2 to 5 carbon atoms,
J represents a C—R₁₄ group or the nitrogen atom
G represents a C—R₁₅ group or the nitrogen atom
R₆, R₁₆ and R₁₇, which are the same or different, represent the hydrogen atom or a lower alkyl group,
R₄, R₅, R₁₄ and R₁₅ taken individually represent the hydrogen atom, a halogen atom, a lower alkyl group, an alkoxy, alkylthio, alkylsulfonyl, alkylsulfoxide, trifluoromethyl, nitro, cyano, carboxy, alkylcarboxy, alkylamino or dialkylamino group,
or, when G or J are not the nitrogen atom, the groups OR₁ and R₁₄ and/or the groups R₁₄ and R₅ and/or the groups R₁₅ and R₅ and/or the groups R₁₅ and R₄ can form, together with the aromatic ring to which they are attached, a ring saturated or not,
   said alkyl, cycloalkyl, aryl, arylalkyl, heteroaryl, heterocycle, heterocycloalkyl, heteroarylalkyl, alkylaminoalkyl, alkoxy, alkoxyalkyl, alkylthio and alkylcarboxy groups, and said ring, being substituted or not,
   and their salts, optical and geometrical isomers or their mixtures.

According to the present invention, the term “alkyl” more particularly denotes a linear or branched hydrocarbon, preferably saturated, having from 1 to 24 carbon atoms. According to the invention, the term “lower alkyl” more particularly denotes a linear or branched hydrocarbon, preferably saturated, having from 1 to 12, preferably from 1 to 6, carbon atoms, such as in particular methyl, ethyl, propyl, isopropyl, butyl, isobutyl, tert-butyl, sec-butyl, pentyl, neopentyl, n-hexyl, heptyl, octyl, nonyl, decyl or dodecyl. When they are branched, the alkyl groups can be selected in particular in the group consisting of the 2-ethylhexyl, 2-methylbutyl, 2-methylpentyl, 1-methylhexyl and 3-methylheptyl groups. C₁-C₄ groups are preferred. Methyl and ethyl groups are more specifically preferred.

According to the invention, the term “halogenoalkyl” designates an alkyl group such as defined hereinabove substituted with at least one halogen atom, preferably fluorine. According to a particular aspect of the invention, R1 represents a perhalogenoalkyl group, preferably perfluoroalkyl, such as trifluoromethyl.

“Alkylene” groups according to the invention are divalent groups corresponding to the alkyl groups defined hereinabove by removal of a hydrogen atom.

Cycloalkyl or cycloalkylene groups are alkyl or alkylene groups such as defined hereinabove forming a ring.

“Aryl” groups are mono-, bi- or tri-cyclic aromatic hydrocarbon systems, generally with 5 or 6 links, containing from 6 to 18 carbon atoms. The phenyl or naphthyl groups are particular examples. Heteroaryl groups are aromatic hydrocarbon systems containing at least one heteroatom, such as in particular nitrogen, sulfur or oxygen, in the ring (or rings).

The term heterocycle denotes mono-, bi- or tri-cyclic aromatic hydrocarbon systems containing at least one heteroatom, such as in particular nitrogen, sulfur or oxygen, in the ring (or rings). Possibly they can have at least one unsaturated bond in the ring (or rings). They can be aromatic or not. Particular examples of heterocycle are the piperidine, piperazine, pyrrolidine, morpholine, homopiperazine, homopiperidine, thiomorpholine, tetrahydropyridine, thiophene, furan, pyridine, pyrimidine, pyridazine and pyrazine groups.

“Alkoxy” groups correspond to alkyl groups defined hereinabove linked to the rest of the molecule by means of an —O— (ether) bond.

The alkyl-COOR₁₇ group corresponds to an alkyl group, preferably a lower alkyl, containing a COOR₁₇ group at the end of the alkyl chain.

“Alkylthio” groups correspond to alkyl groups defined hereinabove linked to the rest of the molecule by means of an —S— (thioether) bond.

“Alkylsulfonyl” groups correspond to alkyl groups defined hereinabove linked to the rest of the molecule by means of a SO2 group.

“Alkylsulfoxide” groups correspond to alkyl groups defined hereinabove linked to the rest of the molecule by means of a SO group.

“Alkylamino” or “dialkylamino” groups correspond to an alkly group or to two alkyl groups such as defined hereinabove linked to the rest of the molecule by a nitrogen atom or an amine group. An alkylaminoalkyl group corresponds to an alkyl group interrupted by an amine group.

“Imidazopyridinylalkyl” groups correspond to the imidazopyridine group linked to the rest of the molecule by an alkyl group such as defined hereinabove.

“Halogen” denotes a fluorine, chlorine, bromine or iodine atom.

“Heteroatom” denotes an atom selected in the group consisting of O, N and S.

Arylalkyl (heteroarylalkyl and heterocycloalkyl) groups are groups comprising an aryl function (heteroaryl and heterocycle, respectively) such as defined hereinabove linked to the rest of the molecule by an alkylene chain. The benzyl and phenethyl groups are particular examples of arylalkyl groups.

“Rings saturated or not” is understood to mean cyclic hydrocarbon systems, aromatic or not, possibly containing heteroatoms and/or unsaturated bonds in their rings. They therefore include in particular aryl, heteroaryl, heterocycle or cycloalkyl groups such as defined hereinabove. Particular examples of rings saturated or not include cycloalkyl, cycloalkylene, piperidine, piperazine, pyrrolidine, morpholine, homopiperazine, homopiperidine, thiomorpholine, and tetrahydropyridine groups.

When the OR₁ and R₁₄ groups and/or the R₅ and R₁₄ groups and/or the R₅ and R₁₅ groups and/or the R₁₅ and R₄ groups form, together with the aromatic ring to which they are attached, a ring saturated or not, it is preferably a ring comprising from 3 to 8 atoms, aromatic or not, possibly containing one or more heteroatoms, preferably from 0 to 3. Preferred examples of said rings are in particular benzofuran, dihydrobenzofuran, benzodioxane, benzopyran, dihydrobenzopyran, benzodioxole.

Furthermore, as indicated earlier, the different groups noted hereinabove may or may not contain one or more substituents, selected for example in the group consisting of halogen, nitro, cyano, trifluoromethyl, carboxy, (C₁-C₆)-alkoxycarbonyl, mono- or di-(C₁-C₆)-alkylaminocarbonyl, aminocarbonyl, mono- or di-(C₆-C₁₂)-aryl- or hetero-(C₂-C₁₂)-arylaminocarbonyl, mono- or di-(C₆-C₁₂)-aryl- or hetero-(C₂-C₁₂)-aryl-(C₁-C₆)-alkylaminocarbonyl, hydroxy, alkoxy, (C₁-C₆)-alkyl, (C₁-C₆)-alkylamino, amino possibly: substituted by one or more groups selected in the group consisting of (C₁-C₆)-alkyl, (C₃-C₈)-cycloalkyl, (C₂-C₁₂)-aryl, hetero-(C₂-C₁₂)-aryl, (C₆-C₁₂)-aryl-(C₁-C₆)-alkyl, hetero-(C₂-C₁₂)-aryl-(C₁-C₆)-alkyl, (C₁-C₇)-alcanoyl, cyclo-(C₃-C₈)-alcanoyl, (C₆-C₁₂)-aroyl, or (C₆-C₁₂)-aryl-(C₁-C₇)-alcanoyl.

Preferred compounds of the invention are compounds represented by formula (I) hereinabove in which at least one of the conditions specified below, preferably all, is met:
A represents a phenyl, a pyrimidine, a pyridazine or a pyrazine and/or
R1 represents a methyl or ethyl group, and/or
n=1 and/or
n′=1 and/or
Y is an alkylene chain having 2 or 3 carbon atoms, preferably linear, and/or
R₂ is a hydrogen atom, and/or
R₃ represents a group selected from among the following: 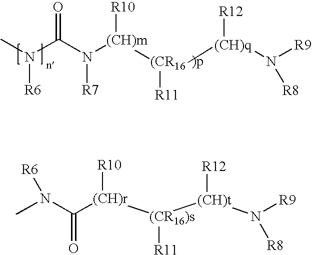
R₄ is a hydrogen atom, and/or
R₆ is a hydrogen atom, and/or
G is a CH group, and/or
J is a CH group.

Other preferred compounds according to the invention are compounds represented by formula (I) hereinabove in which at least one of the conditions specified below, preferably all, is met:
A represents a phenyl, a pyrimidine, a pyridazine or a pyrazine and/or
R1 represents a methyl or ethyl group, and/or
n=1 and/or
n′=0 and/or
Y is an alkylene chain having 2 or 3 carbon atoms, preferably linear, and/or
R₂ is a hydrogen atom, and/or
R₃ represents a group selected from among the following: 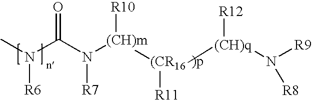
R₄ is a hydrogen atom, and/or
G is a CH group, and/or
J is a CH group.

Other preferred compounds according to the invention are compounds represented by formula (I) hereinabove in which at least one of the conditions specified below, preferably all, is met:
A represents a phenyl, a pyrimidine, a pyridazine or a pyrazine and/or
R1 represents a lower alkyl group, preferably a methyl or ethyl group, and/or
n=1 and/or
Y is an alkylene chain having 2 or 3 carbon atoms, preferably linear, and/or
R₂ is a hydrogen atom, and/or
R₄ is a hydrogen atom, and/or
R₅ is a hydrogen atom, and/or
G is a CH group, and/or
J is a CH group, and/or
R₃ represents a group selected from among the following: 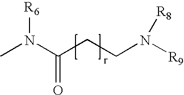
   where R₆ is a hydrogen atom or a lower alkyl group (in particular methyl) and r represents 0, 1 or 2 (in particular 1 or 2).

Other preferred compounds according to the invention are compounds represented by formula (I) hereinabove in which at least one of the conditions specified below, preferably all, is met:
A represents a phenyl, a pyrimidine, a pyridazine or a pyrazine and/or
R1 represents a lower alkyl group, in particular a methyl or ethyl group, and/or
n=1 and/or
Y is an alkylene chain having 2 or 3 carbon atoms, preferably linear, and/or
R₂ is a hydrogen atom, and/or
R₄ is a hydrogen atom, and/or
R₅ is a hydrogen atom, and/or
G is a CH group, and/or
J is a CH group, and/or
R₃ represents a group selected from among the following: 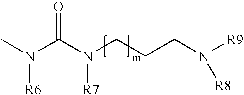
   where R₆ is a hydrogen atom or a lower alkyl group (in particular methyl), R₇ is a hydrogen atom or a lower alkyl group (in particular methyl), and m is an integer comprised between 0 and 2 inclusive (in particular 0 or 1).

Other preferred compounds according to the invention are compounds represented by formula (I) hereinabove in which at least one of the conditions specified below, preferably all, is met:
A represents a phenyl, a pyrimidine, a pyridazine or a pyrazine and/or
R1 represents a lower alkyl group, in particular a methyl or ethyl group, and/or
n=1 and/or
Y is an alkylene chain having 2 or 3 carbon atoms, preferably linear, and/or
R₂ is a hydrogen atom, and/or
R₄ is a hydrogen atom, and/or
R₅ is a hydrogen atom, and/or
G is a CH group, and/or
J is a CH group, and/or
R₃ represents a group selected from among the following: 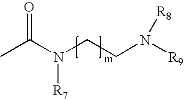
   where R₇ is a hydrogen atom or a lower alkyl group (in particular methyl) and m represents 1 or 2.

A preferred subfamily according to the invention is represented by compounds having formula (I) in which R1 represents a lower alkyl group, in particular methyl or ethyl. As illustrated in the examples, said derivatives according to the invention exhibit advantageous properties as 5-HT4 receptor ligands.

Another particular category of compounds according to the invention is represented by compounds having general formula (I) in which A represents a heterocycle of 6 atoms, possibly substituted, containing one or two nitrogen atoms or a phenyl group possibly substituted.

According to a particularly preferred embodiment, the invention deals with compounds having formula (I) in which Y is an alkylene chain of 2 or 3 carbons, R1 represents a methyl or ethyl group and A represents a phenyl group substituted or not.

Another particularly preferred category of compounds according to the invention is represented by compounds of formula (I) in which R3 represents a —NR₆—COR₁₃ or —(NR₆)_{n′}—CONR₇R₁₃ group, where R₁₃ represents a cycloalkyl group, a heterocycle, an arylalkyl group, a heteroarylalkyl group, a heterocycloalkyl group, an alkylcarboxy group, a cycloalkylcarboxy group, an alkyl-COOR₁₇ group, an imidazopyridinylalkyl group, a trifluoroalkyl group or a heteroarylthioalkyl group.

A particular family according to the invention is represented by compounds having general formula (I) such as defined hereinabove, and the subfamilies indicated hereinabove, in which R₂ is a hydrogen atom, and/or R₄ is a hydrogen atom, and/or R₁₄ is a hydrogen atom, and/or R₁₅ is a hydrogen atom, even more preferably in which at least two of the groups R₂, R₄, R₁₄ and R₁₅ are a hydrogen atom, even more preferably in which the four groups R₂, R₄, R₁₄ and R₁₅ each represent a hydrogen atom.

Another particular family according to the invention is represented by compounds having general formula (I) such as defined hereinabove, and the subfamilies indicated hereinabove, in which G is the CH group and/or J is the CH group, more preferably in which G and J each represent the CH group.

Another particular family according to the invention is represented by compounds having general formula (I) such as defined hereinabove, and the subfamilies indicated hereinabove, in which n equals 1.

A particular family according to the invention is represented by compounds having general formula (I) such as defined hereinabove, and the subfamilies indicated hereinabove, in which Y is an alkylene chain having 2 or 3 carbon atoms, preferably not branched.

Another particular family of compounds according to the invention is represented by compounds having general formula (I) such as defined hereinabove in which R4, R5, R14 and R15 represent the hydrogen atom.

A particular category of compounds according to the invention is represented by compounds having general formula (I) in which at least one of R4, R5, R14 and R15 is different from the hydrogen atom. In particular, at least one of R4, R5, R14 and R15 represents an alkoxy group (in particular methoxy), NO2, alkyl (in particular methyl) or a halogen atom (in particular chlorine or fluorine), the other R4, R5, R14 and R15 advantageously representing a hydrogen atom.

As indicated, the inventive compounds can be in the form of salts, in particular basic or acid addition salts, preferably compatible with a pharmaceutical use.

Among the pharmaceutically acceptable acids, non-limiting examples include hydrochloric, hydrobromic, sulfuric, phosphoric, acetic, trifluoroacetic, lactic, pyruvic, malonic, succinic, glutaric, fumaric, tartaric, maleic, citric, ascorbic, methane or ethane sulfonic, camphoric acid, etc. Among the pharmaceutically acceptable bases, non-limiting examples include sodium hydroxide, potassium hydroxide, triethylamine and tert-butylamine.

Specific examples of preferred compounds of the invention are in particular the compounds described in the examples, more specifically those of examples 9-67, 72-102, 104-106 and 112-119, and the salts thereof, and in particular the following compounds: 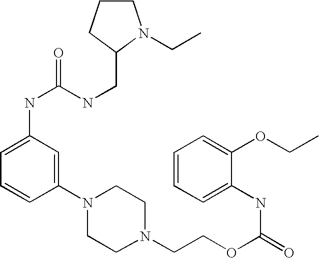
2-(4-{3-[3-(1-ethyl-pyrrolidin-2-ylmethyl)-ureido]-phenyl}-piperazin-1-yl)-ethyl-N-(2-ethoxy-phenyl)carbamate, 57 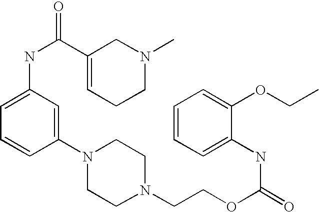
2-(4-{3-[(1-methyl-1,2,5,6-tetrahydro-pyridine-3-carbonyl)-amino]-phenyl}-piperazin-1-yl)-ethyl-N-(2-ethoxy-phenyl)carbamate, 21 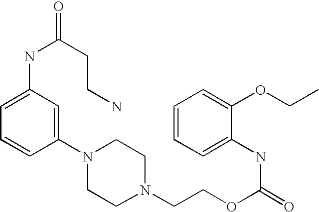
2-{4-[3-(3-amino-propionylamino)-phenyl]-piperazin-1-yl}-ethyl-N-(2-ethoxy-phenyl)carbamate, 42 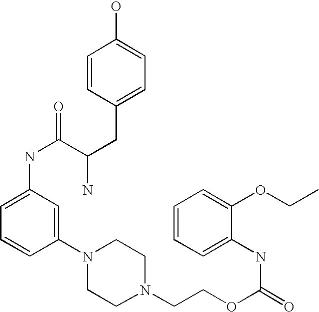
2-(4-{3-[2-amino-3-(4-hydroxy-phenyl)-propionylamino]-phenyl}-piperazin-1-yl)-ethyl-N-(2-ethoxy-phenyl)carbamate, 44 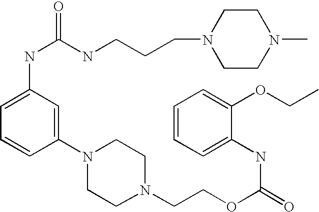
2-[4-(3-{3-[3-(4-methyl-piperazin-1-yl)-propyl]-ureido}-phenyl)-piperazin-1-yl]-ethyl-N-(2-ethoxy-phenyl)carbamate, 60 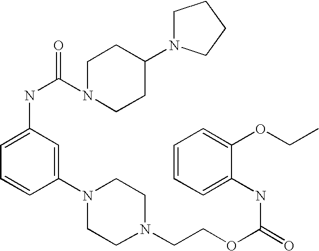
2-(4-{3-[(4-pyrrolidin-1-yl-piperidine-1-carbonyl)-amino]-phenyl}-piperazin-1-yl)-ethyl-N-(2-ethoxy-phenyl)carbamate, 59 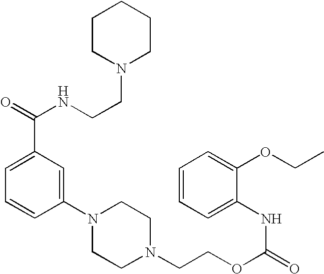
2-(4-{3-[2-piperidin-1-yl-ethylcarbamoyl]-phenyl}-piperazin-1-yl)-ethyl-N-(2-ethoxy-phenyl)carbamate, 78 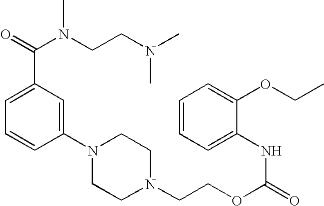
2-(4-{3-[(2-dimethylamino-ethyl)-methyl-carbamoyl]-phenyl}-piperazin-1-yl)-ethyl-N-(2-ethoxy-phenyl)carbamate, 105

The compounds represented by formula (I) can be prepared by methods known to those skilled in the art. In this respect the invention describes different routes of synthesis, which are illustrated in FIGS. 1-4 and in the examples, and which can be practiced by those skilled in the art, as indicated in the examples. The starting compounds are commercially available or can be synthesized by the usual methods. It is understood that the present application is not limited to a particular route of synthesis, and encompasses other methods allowing the production of the indicated compounds.

According to a particular object, the invention is based on a method for preparing a compound such as defined hereinabove, characterized in that a product represented by formula (II) is reacted with a product represented by formula (III): 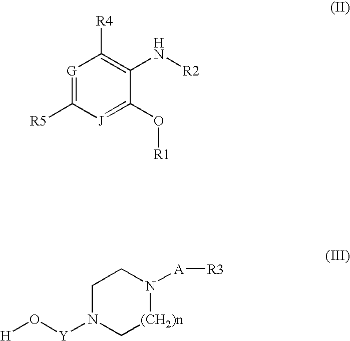
in which the groups R1, R2, R3, R4, R5, A, Y, J, G and n are such as defined hereinabove, in the presence of a carbonyl donor reagent, preferably triphosgene or the (Boc)₂O/DMAP system, and the resulting product is recovered. Advantageously, the reaction is carried out in a solvent, for example neutral, typically an aprotic solvent (see FIG. 1).

According to a particular object, the invention is based on a method for preparing a compound such as defined hereinabove, characterized in that a product represented by formula (I) is reacted with a product represented by formula (III): 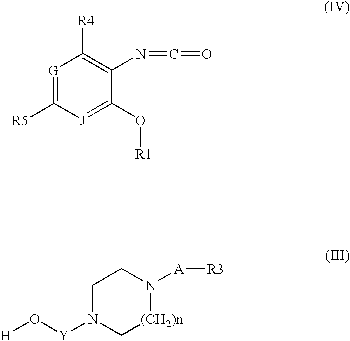
in which the groups R1, R2, R3, R4, R5, A, Y, J, G and n are such as defined hereinabove. Advantageously, the reaction is carried out in a solvent, for example neutral, typically an aprotic solvent (see FIG. 2).

Another particular object of the invention is based on a method for preparing a compound such as defined hereinabove, characterized in that a product represented by formula (V) is reacted with a product represented by formula (VI) in which the groups R1, R2, R3, R4, R5, R6, R7, A, Y, J, G, R8-R12, R16 and n, m, p, q are such as defined hereinabove, in the presence of a carbonyl donor reagent, preferably triphosgene or carbonyl di-imidazole. Advantageously, the reaction is carried out in a solvent, for example neutral, typically an aprotic solvent (see FIG. 3).

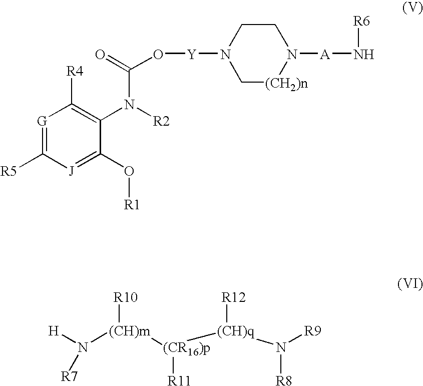

Another particular object of the invention is based on a method for preparing a compound such as defined hereinabove, characterized in that a product represented by formula (V) is reacted with a product represented by formula (VII) in which the groups R1, R2, R3, R4, R5, R6, R7, R13, A, Y, J, G and n are such as defined hereinabove, in the presence of a carbonyl donor reagent, preferably triphosgene or carbonyl di-imidazole. Advantageously, the reaction is carried out in a solvent, for example neutral, typically an aprotic solvent (see FIG. 3).

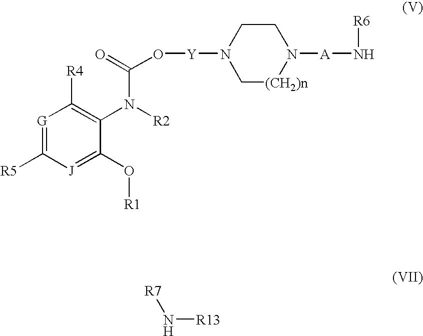

Another particular object of the invention is based on a method for preparing a compound such as defined hereinabove, characterized in that a product represented by formula (V) is reacted with a product represented by formula (VIII) in which the groups R1, R2, R3, R4, R5, R6, R7, A, Y, J, G, R8-R12, R16, n, r, s and t are such as defined hereinabove, in the presence of a classical coupling agent like DCC on solid support, EDCI, PS-carbodiimide resin. Advantageously, the reaction is carried out in a solvent, for example neutral, typically an aprotic solvent (see FIG. 4).

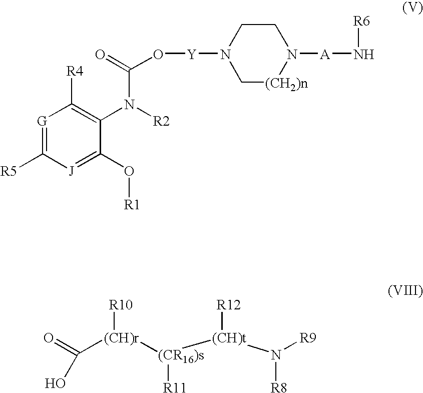

Another particular object of the invention is based on a method for preparing a compound such as defined hereinabove, characterized in that a product represented by formula (V) is reacted with a product represented by formula (IX) in which the groups R1, R2, R3, R4, R5, R6, R7, R13, A, Y, J, G and n are such as defined hereinabove, in the presence of a coupling agent like DCC, EDCI, PS-carbodiimide resin. Advantageously, the reaction is carried out in a solvent, for example neutral, typically an aprotic solvent (see FIG. 4).

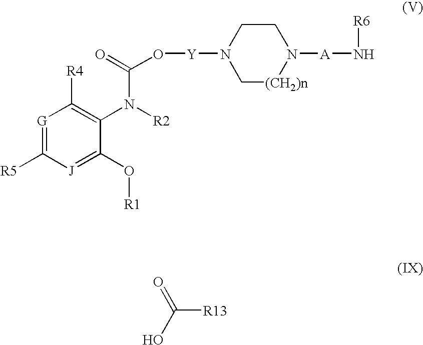

Another particular object of the invention is based on a method for preparing a compound such as defined hereinabove, characterized in that a product represented by formula (X) is reacted with a product represented by formula (VI) in which the groups R1, R2, R3, R4, R5, R7, A, Y, J, G, R8-R12 and n, m, p, q are such as defined hereinabove, in the presence of a coupling agent like DCC, EDCI, PS-carbodiimide resin. Advantageously, the reaction is carried out in a solvent, for example neutral, typically an aprotic solvent (see FIG. 5).

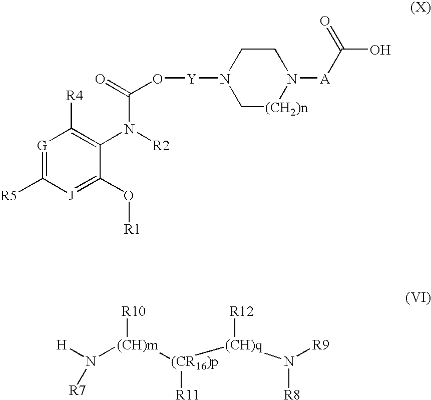

Another particular object of the invention is based on a method for preparing a compound such as defined hereinabove, characterized in that a product represented by formula (X) is reacted with a product represented by formula (VII) in which the groups R1, R2, R3, R4, R5, R7, R13, A, Y, J, G and n are such as defined hereinabove, in the presence of a coupling agent like DCC, EDCI, PS-carbodiimide resin. Advantageously, the reaction is carried out in a solvent, for example neutral, typically an aprotic solvent (see FIG. 5).

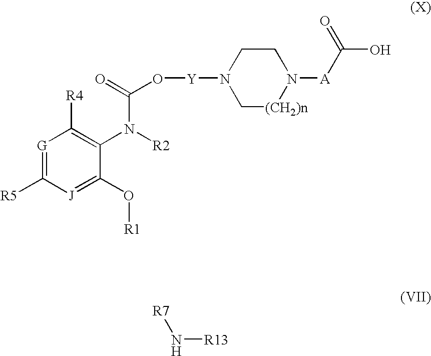

Another object of the invention concerns intermediate products useful for preparing the inventive compounds. Said intermediate products are more particularly selected in the group consisting of ethyl 3-{4-[2-(2-ethoxy-phenylcarbamoyloxy)-ethyl]-piperazin-1-yl}-benzoate, sodium 3-{4-[2-(2-ethoxy-phenylcarbamoyloxy)-ethyl]-piperazin-1-yl}-benzoate and one of the addition salts of same.

Another object of the invention concerns any pharmaceutical composition comprising a compound such as defined hereinabove. Advantageously it is a pharmaceutical composition for the treatment or prophylaxis of diseases involving a 5-HT4 receptor, for example the 5-HT4e receptor. The pharmaceutical compositions according to the invention are useful in particular for the treatment or prophylaxis of gastrointestinal disorders, central nervous system disorders, heart diseases, urological disorders, obsessional behaviors, migraine or pain.

The invention further concerns the use of a compound such as defined hereinabove for preparing a pharmaceutical composition for implementing a method of treatment or prophylaxis of the human or animal body.

The invention further concerns a method for treating a pathology involving a 5-HT4 receptor, comprising administering to a subject, particularly human, an effective dose of a compound or a pharmaceutical composition such as defined hereinabove.

The pharmaceutical compositions according to the invention advantageously comprise one or more pharmaceutically acceptable excipients or vehicles. Examples include saline, physiological, isotonic, buffered solutions and the like, compatible with pharmaceutical use and known to those skilled in the art. The compositions may contain one or more agents or vehicles selected in the group consisting of dispersants, solubilizers, stabilizers, preservatives, and the like. Agents or vehicles that can be used in the formulations (liquid and/or injectable and/or solid) are in particular methylcellulose, hydroxymethylcellulose, carboxymethylcellulose, polysorbate 80, mannitol, gelatin, lactose, plant oils, acacia, and the like. The compositions may be formulated as suspensions for injection, gels, oils, tablets, suppositories, powders, capsules, gelules, and the like, possibly by means of pharmaceutical forms or devices ensuring prolonged and/or delayed release. For this type of formulation, an agent such as cellulose, carbonates or starches is advantageously used.

The inventive compounds or compositions may be administered in different ways and in different forms. For instance, they may be administered by the oral or systemic route, preferably systemic, such as for example by the intravenous, intramuscular, subcutaneous, transdermal, intra-arterial route, etc. For injections, the compounds are generally formulated as liquid suspensions, which can be injected through syringes or by infusion, for example. It is understood that the injection rate and/or the injected dose may be adapted by those skilled in the art according to the patient, the pathology, the method of administration, etc. Typically, the compounds are administered at doses ranging from 0.1 μg to 100 mg/kg of body weight, more generally from 0.01 to 10 mg/kg, typically between 0.1 and 10 mg/kg. In addition, repeated injections may be given, where appropriate. Moreover, the inventive compositions may additionally comprise other active ingredients or agents.

Other aspects and advantages of the invention will become apparent in the following examples, which are given for purposes of illustration and not by way of limitation.

### LEGEND OF FIGURES

FIG. 1: Synthesis scheme 1 of the inventive compounds. A, Y, G, J and the groups R1-R5 are defined as hereinabove.

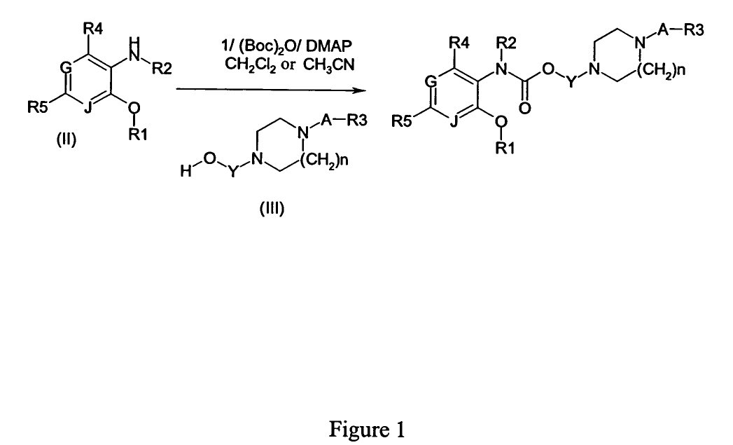

FIG. 2: Synthesis scheme 2 of the inventive compounds. A, Y, G, J and the groups R1 and R3-R5 are defined as hereinabove.

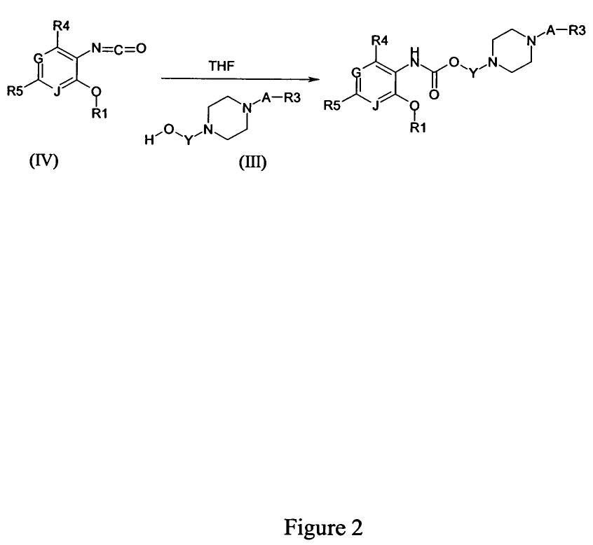

FIG. 3: Synthesis scheme 3 of the inventive compounds. A, Y, G, J and the groups R1-R2 and R4-R13 are defined as hereinabove.

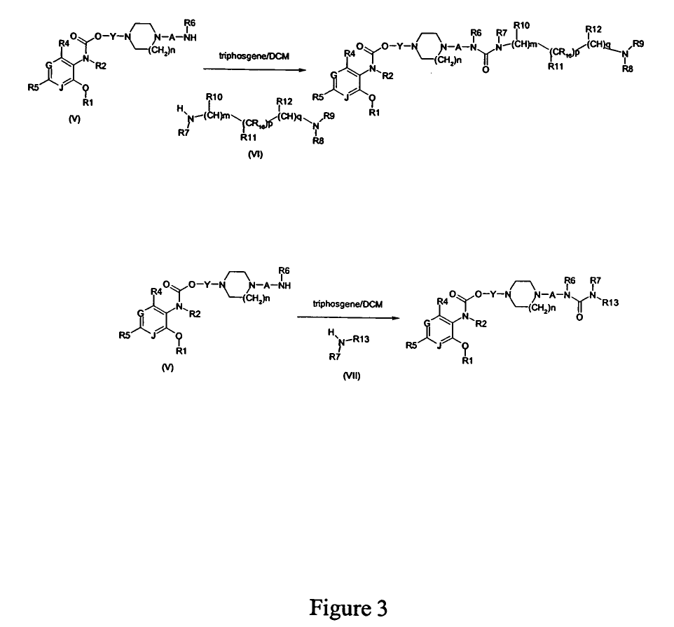

FIG. 4: Synthesis scheme 4 of the inventive compounds. A, Y, G, J and the groups R1-R2, R4-R6 and R8-R13 are defined as hereinabove.

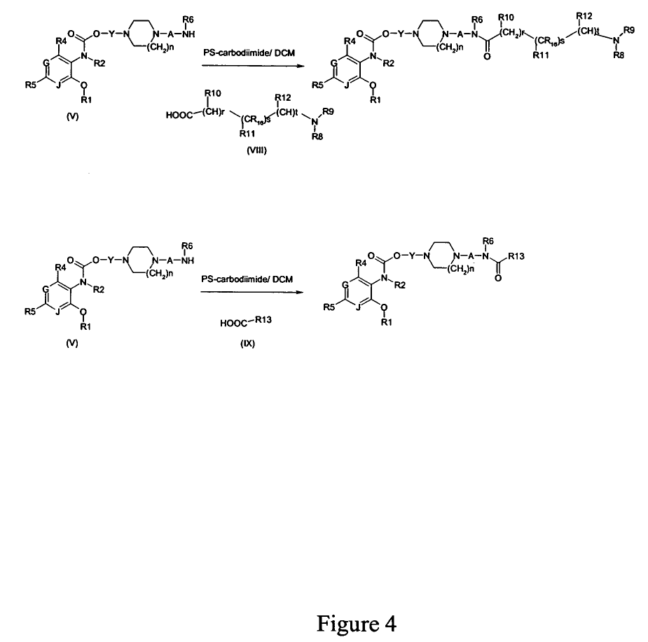

FIG. 5: Synthesis scheme 5 of the inventive compounds. A, Y, G, J and the groups R1-R2, R4-R5 and R7-R13 are defined as hereinabove.

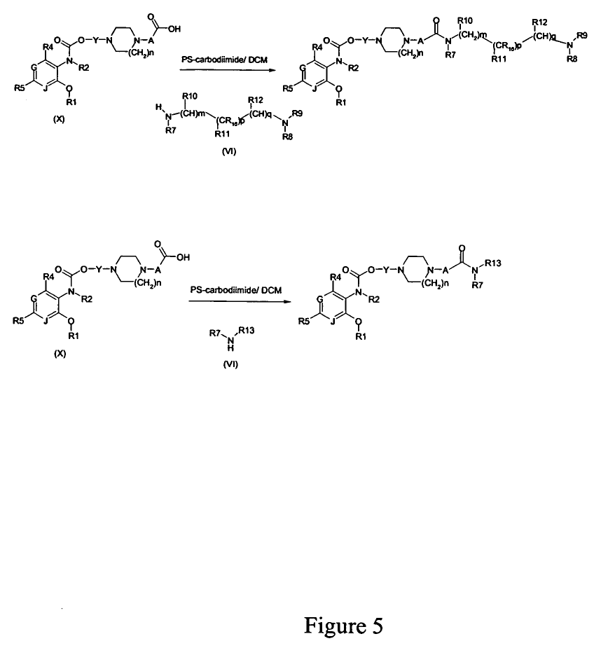

### MATERIALS AND METHODS

The inventive compounds were produced by using conventional methods of parallel synthesis and organic synthesis.

The ¹H and ¹³C NMR spectra were recorded on a Brucker spectrometer model AC-200. Chemical shifts are given in ppm with tetramethylsilane as internal reference. The symbols m, s, sl, d, t, q, quint., dd, td, etc. respectively signify multiplet, singlet, broad singlet, doublet, triplet, quadruplet, quintuplet, split doublet, split triplet. Infrared spectra were recorded on a Perkin Elmer model 841 (KBr disks) or on a Brucker Vector 22 Fourier transformation apparatus.

Melting points were determined on a Kofler apparatus.

Purifications by preparative HPLC were carried out on a VWR-Knauer system with an Uptisphere UP5ODS&10US C18 column (100×28 mm) at a wavelength of 220 nm (flow rate: gradient from 15 to 50 ml/min in 4 min, then constant at 50 ml/min; solvent A=water/acetonitrile/trifluoroacetic acid 95/5/0.05 and solvent B=acetonitrile/water/trifluoroacetic acid 80/20/0.05).

The HPLC chromatograms were recorded on a Shimadzu SCL10A system with an Uptisphere UP50DB-5m C18 column (4.6×50 mm) with a flow rate of 4 ml/min at a wavelength of 220 nm.

HPLC/MS analyses were carried out on a Plateform LC Micromass spectrometer with an APCI ionization source (column TSK gel super ODS 4.6 mm ID×5 cm, flow rate 2.75 ml/min, gradient: 100% A to 100% B in 3 min., plateau of 100% B 1 min, solvent A=water/0.05% trifluoroacetic acid and solvent B=acetonitrile/water/trifluoroacetic acid 80/20/0.05).

Unless otherwise indicated, the products used for preparing the compounds represented by formula (I) were obtained commercially and used without further purification. The following experimental protocols are in no way limiting and are given for purposes of illustration.

### EXAMPLE 1

### 1-(3-nitrophenyl)piperazine

28.4 g (0.33 mol, 5.5 eq) of piperazine were dissolved in 50 ml of DMSO, then 6.4 ml (60 mmol) of 3-fluoronitrobenzene were added. The mixture was heated at 100° C. for 60 h. After cooling, the reaction medium was poured in 530 ml of water. The resulting precipitate was filtered and the filtrate extracted with Et₂O. After drying on MgSO₄ and vacuum concentration, the crude product was purified by column chromatography (eluent: CH₂Cl₂/MeOH 9:1) to give 8.04 g of the expected compound in the form of an orange oil which crystallized at room temperature (yield: 65%).

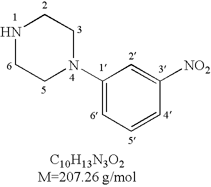

¹H NMR (CDCl₃) δ (ppm): 7.70 (t, Jₘ=2 Hz, 1H, CH_{(2′)}); 7.64 (ddd, Jₒ=8 Hz, Jₘ=2 Hz, Jₘ=0.6 Hz, 1H, CH_{(4′)}); 7.36 (t, Jₒ=8 Hz, 1H, CH_{(5′)}), 7.17 (ddd, Jₒ=8 Hz, Jₘ=2 Hz, Jₘ=0.6 Hz, 1H, CH_{(6′)}); 3.30-3.18 (m, 4H, CH₂₍₃₎, CH₂₍₅₎); 3.09-2.97 (m, 4H, CH₂₍₂₎, CH₂₍₆₎).

### EXAMPLE 2

### 2-bromoethyl-N-(2-ethoxyphenyl)carbamate

In a 50 ml double-neck flask placed in an argon atmosphere, 1.5 g (7 mmol, 1.4 eq) of (Boc)₂O was dissolved in 5 ml of anhydrous CH₂Cl₂, after which were added dropwise 61 mg (0.5 mol, 10% eq) of DMAP dissolved in 5 ml of anhydrous CH₂Cl₂ followed by 0.65 ml (5 mmol) of 2-ethoxyaniline. The mixture was stirred for 20 min at room temperature before adding 0.5 ml (7 mmol, 1.4 eq) of 2-bromoethanol diluted in 5 ml of anhydrous CH₂Cl₂. The reaction was left for 30 min at room temperature then for about 15 h under dichloromethane reflux. The medium was then cooled and vacuum concentrated. The residue obtained was purified by silica gel column chromatography (eluent: CH₂Cl₂) to give 1.43 g of a colorless oil (yield: 99%).

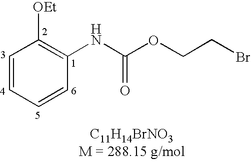

¹H NMR (CDCl₃) δ (ppm): 8.06 (dd, J=8 Hz, J=2 Hz, 1H, CH₍₆₎); 7.31 (sl, 1H, NH); 7.04-6.80 (m, 3H, CH₍₃₎, CH₍₄₎, CH₍₅₎); 4.49 (t, J=7 Hz, 2H, CH₂O); 4.10 (q, J=7 Hz, 2H, OCH₂); 3.59 (t, J=7 Hz, 2H, CH₂Br); 1.49 (t, J=7 Hz, 3H, CH₃).

¹³C NMR (CDCl₃) δ (ppm): 152.7 (CO); 146.9 (C₂); 127.3 (C₁); 123.0 (C₄); 120.9 (C₆); 118.3 (C₅); 110.9 (C₃); 64.4 (CH₂O); 64.2 (CH₂O); 29.1 (CH₂Br); 14.8 (CH₃).

GC (RT, min, 90° C., 1 min, 10° C./min, 150° C., 20° C./cm): 9.91.

### EXAMPLE 3

### 2-[4-(3-nitrophenyl)piperazino]ethyl-N-(2-ethoxyphenyl)carbamate

Reaction between 0.47 g (1.62 mmol) of the compound of example 2.282 μl (1.62 mmol, 1 eq) of DIEA and 0.42 g (1.46 mmol, 0.9 eq) of the compound of example 1 and a few KI crystals in 5 ml of DMF led, after treatment, to an oil which was purified by column chromatography (eluent: EtOAc/petroleum ether, 8:2) to give a mixture which was again purified by column chromatography (eluent: CH₂Cl₂ then CH₂Cl₂:MeOH 9:1). The product is an orange oil stored as the hydrochloride. The reaction produced 384 mg of an orange solid (yield: 58%).

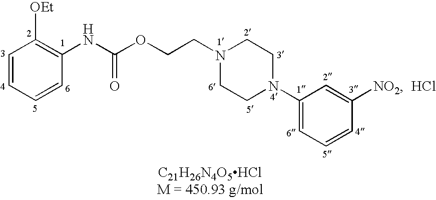

¹H NMR base (CDCl₃) δ (ppm): 8.10 (dd, Jₒ=6 Hz, Jₘ=3 Hz, 1H, CH₍₆₎); 7.70 (t, Jₘ=2 Hz, 1H, CH_{(2″)}); 7.64 (ddd, Jₒ=8.2 Hz, Jₘ=2 Hz, Jₘ=0.8 Hz, 1H, CH_{(4″)}); 7.36 (t, Jₒ=8.2 Hz, 1H, CH_{(5″)}), 7.30 (sl, 1H, NH); 7.17 (ddd, J₀=8.2 Hz, Jₘ=2 Hz, Jₘ=0.8 Hz, 1H, CH_{(6″)}); 7.03-6.80 (m, 3H, CH₍₃₎, CH₍₄₎, CH₍₅₎); 4.35 (t, J=6 Hz, 2H, OCH₂); 4.09 (q, J=7 Hz, 2H, OCH₂); 3.56-3.26 (m, 4H, CH_{2(3′)}, CH_{2(5′)}); 2.77 (t, J=6 Hz, 2H, CH₂N); 2.75-2.64 (m, 4H, CH_{2(2′)}, CH_{2(6′)}); 1.45 (t, J=7 Hz, 2H, CH₃).

¹³C NMR base (CDCl₃) δ (ppm): 153.2 (CO₂); 151.7 (C_{1″}); 149.2 (C_{3″}); 146.8 (C₂); 129.5 (C_{5″}); 127.5 (C₁); 122.7 (2C, C₄, C_{4″}); 120.9 (C₆); 118.2 (C₅); 113.5 (C_{6″}); 110.8 (C₃); 109.5 (C_{2″}); 64.0 (CH₂O); 61.9 (CH₂O); 56.9 (CH₂N); 52.9 (2C, C_{2′}, C_{6′}); 48.2 (2C, C_{3′}, C_{5′}); 14.8 (CH₃).

Elemental Analysis:

| | Calc. (¾H₂O) | Exp. |
|---|---|---|
| % C | 54.30 | 54.31 |
| % H | 6.18 | 6.30 |
| % N | 12.06 | 11.81 |

### EXAMPLE 4

### 2-[4-(3-aminophenyl)piperazinol]ethyl-N-(2-ethoxyphenyl)carbamate

1.08 g (2.60 mmol) of the compound of example 3 was dissolved in 40 ml of methanol. A spatula tip of Raney nickel was added and the mixture was placed in a hydrogen atmosphere and stirred at room temperature. The progress of the reaction was monitored by TLC (mobile phase: EtOAc/petroleum ether 8:2). After 30 min the reaction medium became decolored. The mixture was then filtered on celite and the filtrate was concentrated under reduced pressure. The crude product was taken up in ether, extracted with aqueous 1 M HCl solution. The extracted aqueous phase was then adjusted to alkaline pH by addition of K₂CO₃ then extracted with EtOAc. After drying on Na₂SO₄ and evaporation of the organic phase, 0.8 g of a pure yellow solid was obtained (yield: 80%).

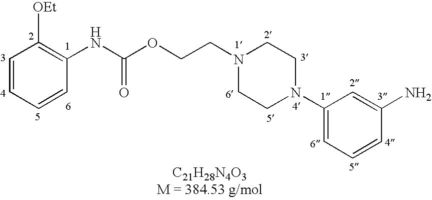

¹H NMR (CDCI₃) δ (ppm): 8.09 (dd, Jₒ=6 Hz, Jₘ=3 Hz, 1H, CH₍₆₎); 7.31 (sl, 1H, NH); 7.10-6.79 (m, 4H, CH₍₃₎, CH₍₄₎, CH₍₅₎, ArH); 6.39-6.17 (m, 3H, ArH); 4.35 (t, J=6 Hz, 2H, OCH₂); 4.09 (q, J=7 Hz, 2H, OCH₂); 3.59 (sl, 2H, NH₂); 3.25-3.14 (m, 4H, CH_{2(3′)}, CH_{2(5′)}); 2.76 (t, J=6 Hz, 2H, CH₂N); 2.70-2.60 (m, 4H, CH_{2(2′)}, CH_{2(6′)}); 1.45 (t, J=7 Hz, 2H, CH₃).

¹³C NMR (CDCl₃) δ (ppm): 153.4 (CO₂); 152.5 (C_{1″}); 147.3 (C_{3″}); 146.9 (C₂); 129.9 (C_{5″}); 127.7 (C₁); 122.7 (C₄); 120.9 (C₆); 118.3 (C₅); 110.9 (C₃); 107.0 (C_{4″}); 106.9 (C_{6″}); 102.9 (C_{2″}); 64.0 (CH₂O); 62.0 (CH₂O); 57.0 (CH₂N); 53.4 (2C, C_{2′}, C_{6′}); 48.8 (2C, C_{3′}, C_{5′}); 14.8 (CH₃).

Elemental Analysis:

| | Calc. (¼H₂O) | Exp. |
|---|---|---|
| % C | 64.84 | 64.93 |
| % H | 7.38 | 7.37 |
| % N | 14.40 | 14.23 |

### EXAMPLE 5

### 2-amino-6-chloropyrazine

120 ml of water, 20.8 ml (0.31 mol, 4.6 eq) of an aqueous 28% ammonia solution and 10 g (0.067 mol, 1 eq) of 2,6-dichloropyrazine were successively added to an autoclave reactor. The autoclave was sealed and the mixture heated at 140° C. for 3 h, then left at room temperature for 60 h. The mixture was filtered, the precipitate washed with water, then vacuum dried. The reaction produced 5.4 g of a fine powder (yield: 63%).

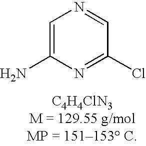

¹H NMR (CDCl₃) δ (ppm): 7.88 (s, 1H, CH); 7.84 (s, 1H, CH); 4.68 (m, 2H, NH₂).

HPLC: t=1.07 min

MS: 130 (MH⁺)

HPLC purity: 100%

### EXAMPLE 6

### 1-(6-chloro-pyrazin-2-yl)-3-ethyl-urea

At 0° C. in a nitrogen atmosphere, 5.5 g (42.5 mmol, 1 eq) of the compound of example 5 were placed in 40 ml of tetrahydrofuran, then 1.79 g (46.7 mmol, 1.1 eq) of a 60% suspension of sodium hydride in mineral oil were added incrementally. The mixture was stirred and the temperature brought to 30° C., until no more hydrogen was given off. After cooling to room temperature, a solution of 5.1 ml (63.8 mmol, 1.5 eq) of ethyl isocyanate in 10 ml of tetrahydrofuran was added dropwise. The mixture was stirred for 16 h at room temperature, then vacuum evaporated. The residue was taken up in a water/ethyl acetate mixture, triturated for 1 h and filtered. The precipitate was washed with ethyl acetate. The reaction produced 7.9 g of a brown solid (yield: 93%).

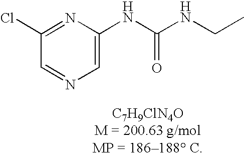

¹H NMR (CDCl₃) δ (ppm): 8.23 (s, 1H, CH); 8.14 (s, 1H, CH); 3.44 (q, J=7 Hz, 2H, NCH₂); 1.26 (t, J=7 Hz, 3H, CH₃).

HPLC: t=1.43 min

MS: 201 (MH⁺)

HPLC purity: 100%

### EXAMPLE 7

### 1-ethyl-3-[4-(2-hydroxy-ethyl)-3,4,5,6-tetrahydro-2H-[1,2′]bipyrazinyl-6′-yl]-urea

624 μl (5 mmol, 1 eq) of N-(2-hydroxyethyl)piperazine and 422 mg (4 mmol, 0.8 eq) of sodium carbonate were added to a suspension of 1 g (5 mmol, 1 eq) of the compound of example 6 in 25 ml of n-butanol. The mixture was refluxed for 36 h, then filtered on a fritted filter. The filtrate was vacuum concentrated. The residue was loaded on a silica chromatography column (eluent: CH₂Cl₂/MeOH 9:1) and produced 0.32 g of an oil which crystallized in air (yield: 22%).

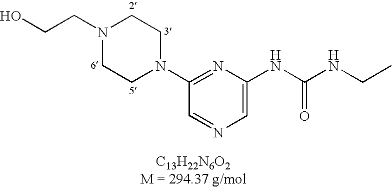

¹H NMR (CDCl₃) δ (ppm): 8.82 (sl, 1H, NH), 8.63 (sl, 1H, NH); 7.67 (s, 1H, CH); 7.59 (s, 1H, CH); 3.68 (t, J=5 Hz, 2H, OCH₂); 3.45-3.65 (m, 4H, CH_{2(3′)}, CH_{2(5′)}); 3.40 (q, J=7 Hz, 2H, NCH₂CH₃); 2.55-2.70 (m, 6H, CH₂CH₂N, CH_{2 (2′)}, CH_{2(6′)}); 2.30 (m, 1H, OH); 1.21 (t, J=7 Hz, 3H, CH₃).

HPLC: t=0.95 min

MS: 295 (MH⁺)

### EXAMPLE 8

### (2-ethoxy-phenyl)-carbamic Acid 2-[6′-(3-ethyl-ureido)-2,3,5,6-tetrahydro-[1,2′]bipyrazinyl-4-yl]-ethyl Ester

318 μl (2.1 mmol, 2 eq) of 2-ethoxyphenyl isocyanate were added to a solution of 310 mg (1.05 mmol, 1 eq) of the compound of example 7 in 5 ml of tetrahydrofuran. The mixture was refluxed for 17 h, then vacuum concentrated. The residue was loaded on a silica chromatography column (eluent: CH₂Cl₂ 95/MeOH 5+0.5% NH₄OH) and produced 0.31 g of a white solid (yield: 65%).

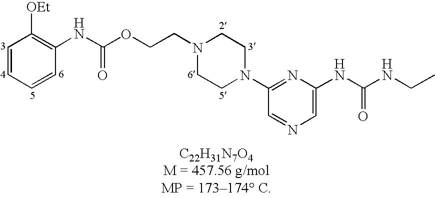

¹H NMR (CDCl₃) δ (ppm): 8.65 (t, J=5 Hz, 1H, NHCH₂); 8.04 (d, J=6 Hz, 1H, CH₍₆₎); 7.65 (s, 1H, CH pyr); 7.62 (s, 1H, CH pyr); 7.32 (sl, 1H, NHCOO); 7.00-6.75 (m, 3H, CH₍₃₎, CH₍₄₎, CH₍₅₎); 4.32 (t, J=6 Hz, 2H, COOCH₂); 4.05 (q, J=7 Hz, 2H, OCH₂CH₃); 3.40-3.55 (m, 4H, CH_{2(3′)}, CH_{2(5′)}); 3.38 (q, J=6 Hz, 2H, NCH₂CH₃); 2.73 (t, J=6 Hz, 2H, CH₂CH₂N); 2.60-2.70 (m, 4H, CH_{2(2′)}, CH_{2(6′)}); 1.41 (t, J=7 Hz, 3H, OCH₂CH₃); 1.19 (t, J=7 Hz, 3H, NCH₂CH₃).

HPLC: t=1.40 min

MS: 458 (MH⁺)

HPLC purity: 100%

### EXAMPLES 9 TO 46

### Amides Derived from Example 4

To a suspension of PS-carbodiimide (Argonaut, 0.96 mmol/g, 74 mg, 0.071 mmol) in 0.33 ml of hydroxyazabenzotriazole solution (153 mM DCM {25% DMF}, 0.05 mmol) were added 0.5 ml of a solution of the compound of example 4 (60 mM DCM {20% DMF}, 0.03 mmol) and 0.5 ml of the carboxylic acid solutions (66 mM DCM {20% DMF}, 0.033 mmol). After 24 h at room temperature, the reaction mixtures were treated overnight with PS-trisamine resin (Argonaut, 3.65 mmol/g, 65 mg, 0.24 mmol). The compounds were obtained after concentrating the filtered solutions to dryness.

The compound of example 26 was also purified by reverse phase HPLC (gradient of CH₃CN/H₂O/TFA: 5/95/0.05 to CH₃CN/H₂O/TFA: 80/20/0.05).

For the protected N-t-butyloxycarbonyl (NHBoc) carboxylic acids (Examples 42 to 46), the compounds were obtained after hydrolysis with 1 ml of DCM/TFA solution (1:1) for 2 h at room temperature and purification on cation exchange resin (SCX).

| | | | Qty | | | | |
|---|---|---|---|---|---|---|---|
| n° | MOLSTRUCTURE | MW | (mg) | Purity % | RT (min) | m | Ion |
| 9 | 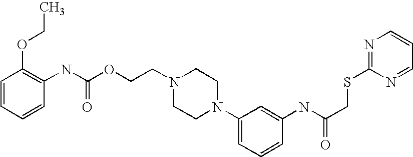 | 536.654 | 2.1 | 68 | 1.58 | 537.61 | M + 1 |
| 10 | 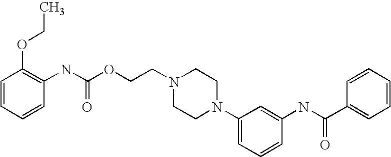 | 488.585 | 11.5 | 94.8 | 1.65 | 489.57 | M + 1 |
| 11 | 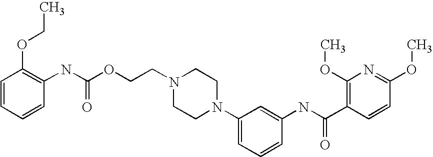 | 549.625 | 10.6 | 96.6 | 1.76 | 550.68 | M + 1 |
| 12 | 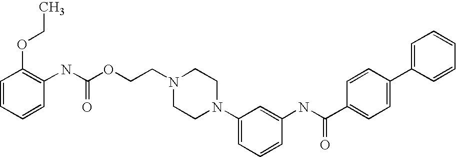 | 564.682 | 10.3 | 100 | 1.85 | 565.65 | M + 1 |
| 13 | 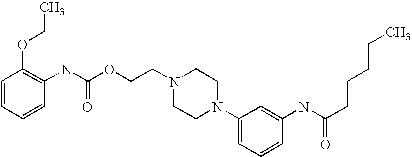 | 482.621 | 14.8 | 96.8 | 1.7 | 483.6 | M + 1 |
| 14 | 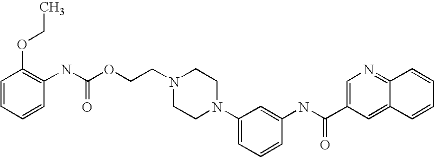 | 539.633 | 15.2 | 96.2 | 1.55 | 540.71 | M + 1 |
| 15 | 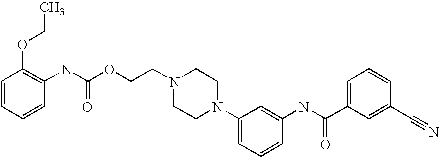 | 513.595 | 15.4 | 89.1 | 1.63 | 514.64 | M + 1 |
| 16 | 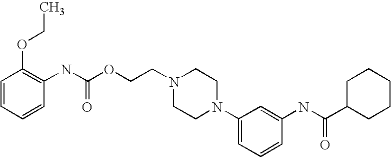 | 494.632 | 14 | 95.8 | 1.7 | 495.65 | M + 1 |
| 17 | 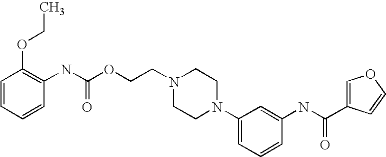 | 478.546 | 12.6 | 95.4 | 1.58 | 479.54 | M + 1 |
| 18 | 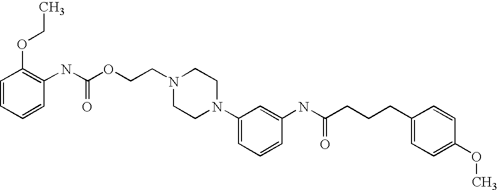 | 560.691 | 17.3 | 97.8 | 1.73 | 561.71 | M + 1 |
| 19 | 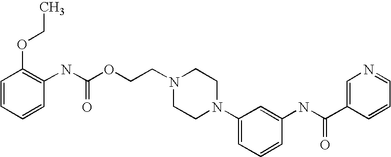 | 489.573 | 13.6 | 98.6 | 1.4 | 490.56 | M + 1 |
| 20 | 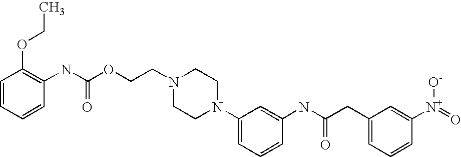 | 547.609 | 10.7 | 92.6 | 1.67 | 548.68 | M + 1 |
| 21 | 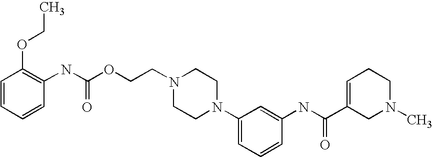 | 507.631 | 15.1 | 96.9 | 1.32 | 508.67 | M + 1 |
| 22 | 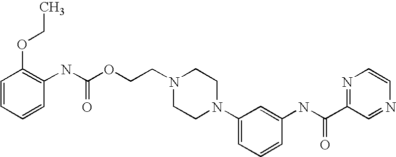 | 490.561 | 9.9 | 95.7 | 1.52 | 491.56 | M + 1 |
| 23 | 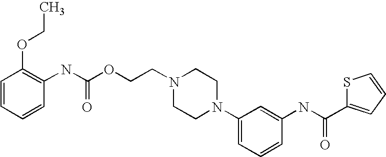 | 494.613 | 13 | 96.8 | 1.62 | 495.55 | M + 1 |
| 24 | 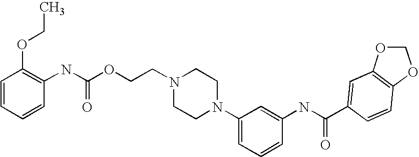 | 532.594 | 5.3 | 93.5 | 1.64 | 533.7 | M + 1 |
| 25 | 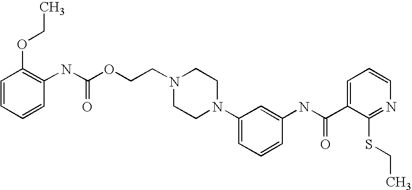 | 549.693 | 15 | 97.9 | 1.68 | 550.67 | M + 1 |
| 26 | 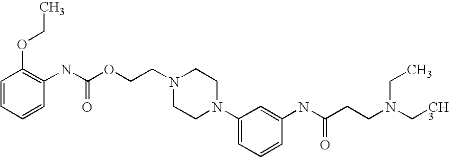 | 511.663 | 5 | 93.3 | 1.34 | 512.74 | M + 1 |
| 27 | 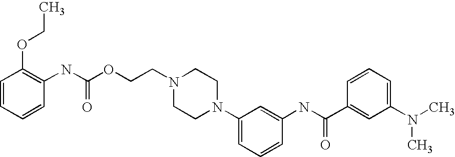 | 531.653 | 11.8 | 90.5 | 1.5 | 532.75 | M + 1 |
| 28 | 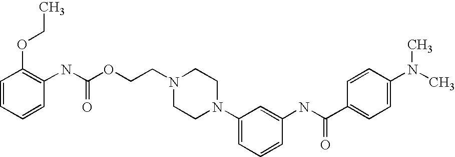 | 531.653 | 8.8 | 95.8 | 1.65 | 532.74 | M + 1 |
| 29 | 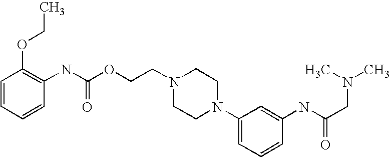 | 469.582 | 13.8 | 100 | 1.3 | 470.61 | M + 1 |
| 30 | 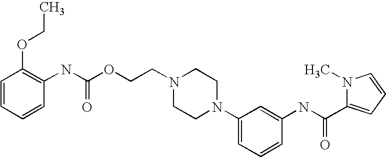 | 491.589 | 9.1 | 96.5 | 1.64 | 492.58 | M + 1 |
| 31 | 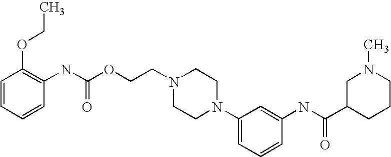 | 509.647 | 17 | 90.4 | 1.32 | 510.7 | M + 1 |
| 32 | 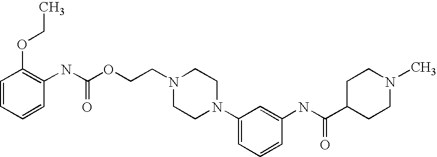 | 509.647 | 17.8 | 93.3 | 1.3 | 510.69 | M + 1 |
| 33 | 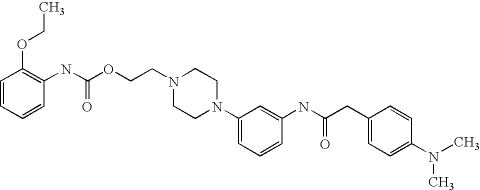 | 545.68 | 16.9 | 94.1 | 1.37 | 546.72 | M + 1 |
| 34 | 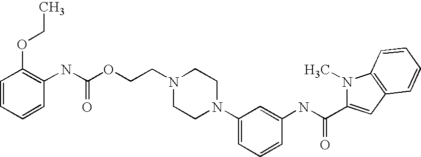 | 541.648 | 14.6 | 95.4 | 1.78 | 542.7 | M + 1 |
| 35 | 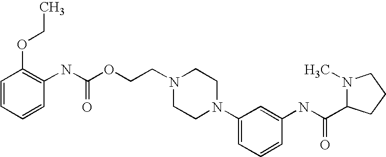 | 495.62 | 13.6 | 93.1 | 1.32 | 496.62 | M + 1 |
| 36 | 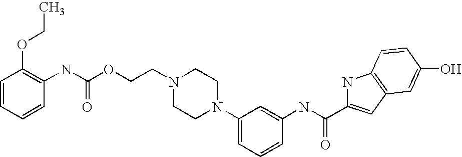 | 543.621 | 10.6 | 88.6 | 1.57 | 544.69 | M + 1 |
| 37 | 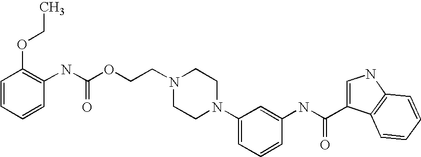 | 527.622 | 4.5 | 53.1 | 1.65 | 528.72 | M + 1 |
| 38 | 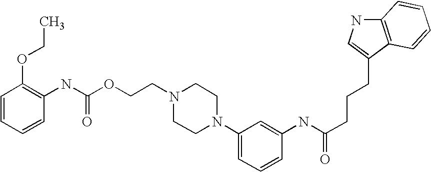 | 569.702 | 19.9 | 88.7 | 1.73 | 570.66 | M + 1 |
| 39 | 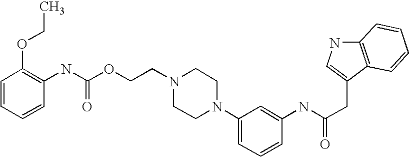 | 541.648 | 4 | 80.6 | 1.65 | 542.72 | M + 1 |
| 40 | 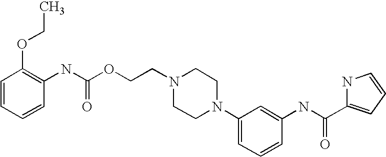 | 477.562 | 13.9 | 83.8 | 1.58 | 478.53 | M + 1 |
| 41 | 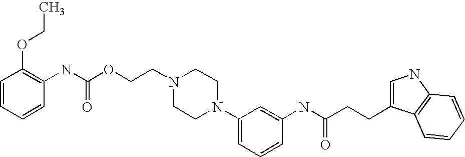 | 555.675 | 18.9 | 92.5 | 1.68 | 556.69 | M + 1 |
| 42 | 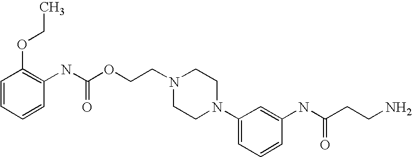 | 455.556 | 12.1 | 95.3 | 1.29 | 456.53 | M + 1 |
| 43 | 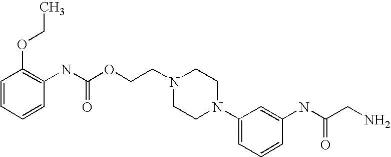 | 441.529 | 6.7 | 86.4 | 1.28 | 442.57 | M + 1 |
| 44 | | 547.652 | 7.7 | 92 | 1.35 | 548.65 | M + 1 |

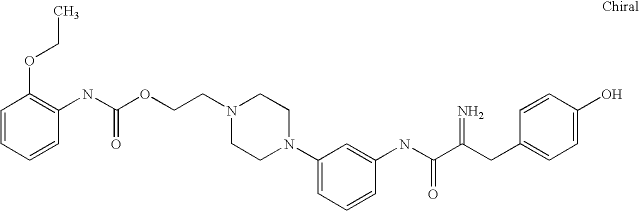

| | | | | | | | |
|---|---|---|---|---|---|---|---|
| 45 | 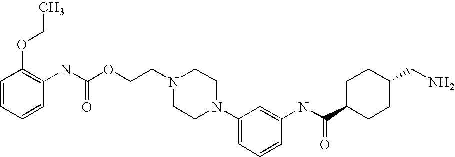 | 523.674 | 11.1 | 100 | 1.34 | 525.35 | M + 1 |
| 46 | 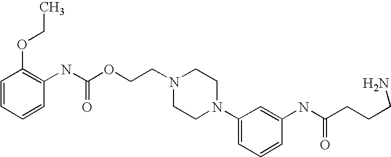 | 489.582 | 11.1 | 94.9 | 1.3 | 470.59 | M + 1 |

### EXAMPLES 47 TO 67

### Ureas Derived from Example 4

A mixture of example 4 (0.45 g, 1.17 mmol) and TEA (489 μl, 3.51 mmol) in 3 ml of DCM was added dropwise at 0° C. to a solution of triphosgene (0.128 g, 0.43 mmol) in 3.3 ml of DCM. The mixture was left to return to room temperature for 1 h, then 0.2 of said mixture was added to the amine solutions (60 mM, 0.06 mmol). The reaction mixtures were left overnight at room temperature, then treated overnight with 0.125 g of PS-Isocyanate (Argonaut, 1.44 mmol/g, 0.12 mmol) and purified on cation exchange resin (SCX).

Some compounds were also purified on silica, eluent A (EtOAc/DCM 80:20) for examples 48, 49, 51 and 53 and eluent B (EtOAc then EtOAc/MeOH 1:1) for examples 54 to 61, or by reverse phase HPLC (gradient of CH₃CN/H₂O/TFA: 5/95/0.05 to CH₃CN/H₂O/TFA: 80/20/0.05) for examples 62 to 67.

| | | | Qty | | | | |
|---|---|---|---|---|---|---|---|
| n° | MOLSTRUCTURE | MW | (mg) | Purity % | RT (min) | m | Ion |
| 47 | 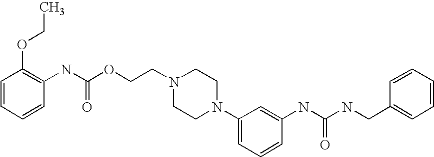 | 517.627 | 12.9 | 92.7 | 1.63 | 518.89 | M + 1 |
| 48 | 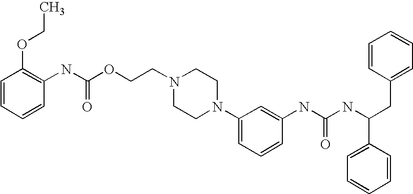 | 607.751 | 5.5 | 100 | 1.82 | 608.86 | M + 1 |
| 49 | 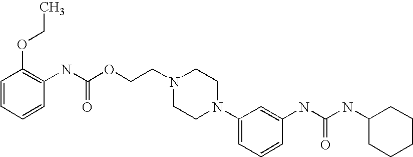 | 509.647 | 3.2 | 100 | 1.68 | 510.61 | M + 1 |
| 50 | 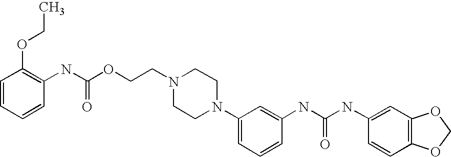 | 547.609 | 14.5 | 90.7 | 1.63 | 548.64 | M + 1 |
| 51 | 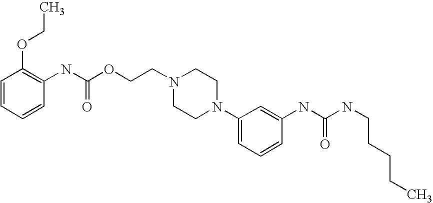 | 497.636 | 3.2 | 100 | 1.67 | 498.61 | M + 1 |
| 52 | 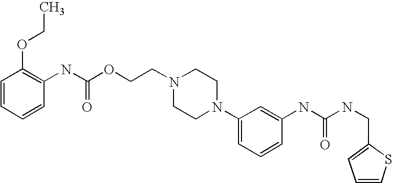 | 523.655 | 11.4 | 96 | 1.61 | 524.67 | M + 1 |
| 53 | 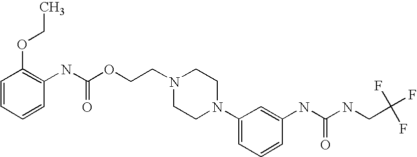 | 509.526 | 4.5 | 100 | 1.58 | 510.59 | M + 1 |
| 54 | 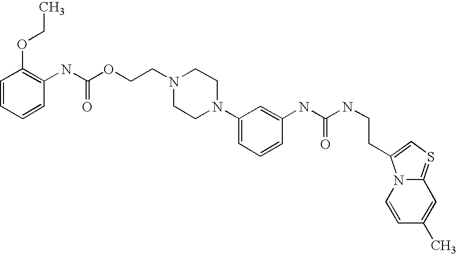 | 585.705 | 10.6 | 74.6 | 1.37 | 586.6 | M + 1 |
| 55 | 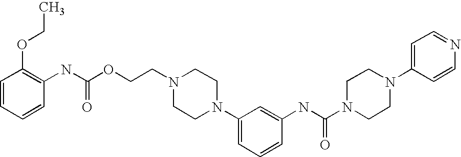 | 573.694 | 1.4 | 80.2 | 1.35 | 574.51 | M + 1 |
| 56 | 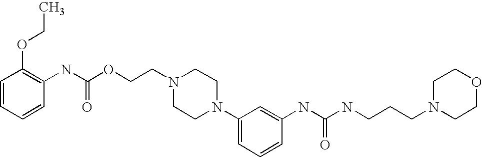 | 554.688 | 1 | 75 | 1.32 | 555.88 | M + 1 |
| 57 | 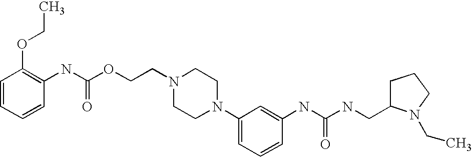 | 538.689 | 1.7 | 83.3 | 1.35 | 539.68 | M + 1 |
| 58 | 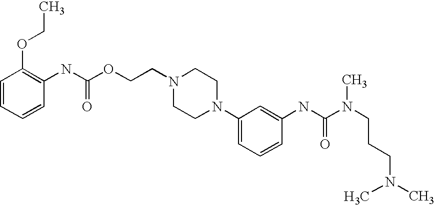 | 526.678 | 1.6 | 89.5 | 1.33 | 527.81 | M + 1 |
| 59 | 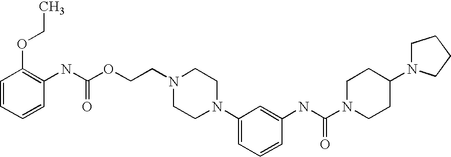 | 564.727 | 2.3 | 68 | 1.34 | 565.67 | M + 1 |
| 60 | 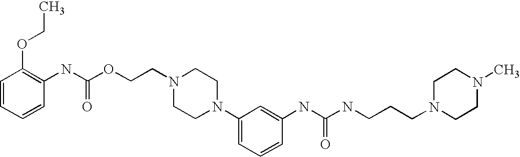 | 567.731 | 2.4 | 80 | 1.26 | 569.06 | M + 1 |
| 61 | 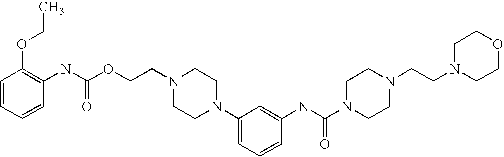 | 609.767 | 2.6 | 84.9 | 1.28 | 610.75 | M + 1 |
| 62 | 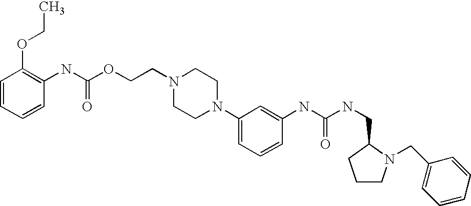 | 600.76 | 3 | 97 | 1.48 | 602.11 | M + 1 |
| 63 | 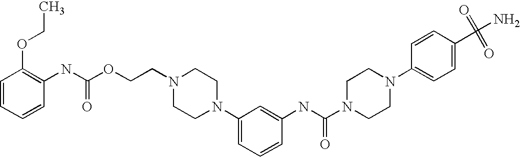 | 651.785 | 5.8 | 69 | 1.51 | 652.48 | M + 1 |
| 64 | 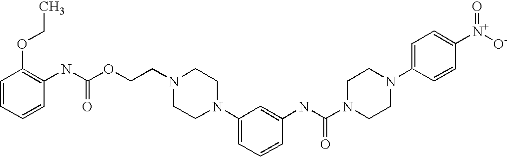 | 617.703 | 8.1 | 94 | 1.7 | 618.68 | M + 1 |
| 65 | 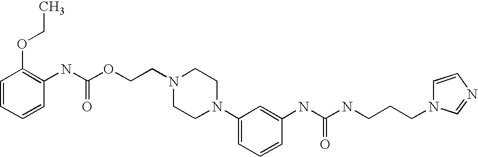 | 535.645 | 6.9 | 96 | 1.32 | 536.65 | M + 1 |
| 66 | 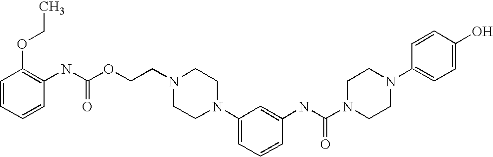 | 588.705 | 8.5 | 95 | 1.39 | 589.66 | M + 1 |
| 67 | 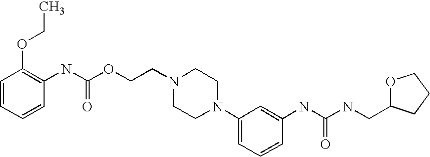 | 511.619 | 4.1 | 95 | 1.5 | 512.48 | M + 1 |

### EXAMPLE 68

### 3-[4-(2-hydroxyethyl)-piperazin-1-yl]-benzonitrile

104 g (0.80 mol, 5.5 eq) of 2-(piperazin-1-yl)ethanol were dissolved in 140 ml of DMSO, then 15.8 ml (0.146 mol) of 3-fluorobenzonitrile were added. The mixture was heated at 100° C. for 90 h. After cooling, the reaction mixture was poured into 1.5 l of water, then extracted with 3×200 ml of ethyl acetate. The pooled organic phases were reextracted with 2×100 ml of aqueous 2 N HCl solution. The acidic aqueous phases were pooled, basified with 100 ml of 4 N sodium hydroxide, then by addition of potassium carbonate until reaching saturation. The mixture was extracted with 3×300 ml of ethyl acetate. The organic phases were pooled, dried on Na₂SO₄ and vacuum concentrated: 18.6 g of the expected compound were obtained as a yellow oil which crystallized at room temperature (yield: 55%).

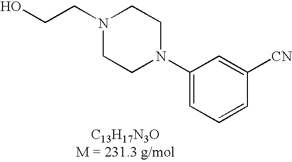

¹H NMR (CDCl₃) δ (ppm): 2.50-2.80 (m, 6H, CH₂CH₂O, CH₂N piperazine); 3.10-3.30 (m, 4H, CH₂N piperazine); 3.67 (t, J=5 Hz, 2H, CH₂O); 7.05-7.15 (m, 3H, Ar—H); 7.25-7.30 (m, 1H, Ar—H).

### EXAMPLE 69

### Ethyl-3-[4-(2-hydroxy-ethyl)-piperazin-1-yl]-benzoate

78.9 ml (1.45 mol) of sulfuric acid were added very slowly to a solution of 18.6 g (80 mmol) of the compound of example 68 in 185 ml of 95° C. ethanol cooled to 0° C. The mixture was refluxed for 6 h, then left at room temperature for 14 h, and then heated under reflux for another 6 h. At 0° C., the mixture was slowly poured into a solution of 154 g of Na₂CO₃ in 300 ml of water. Sodium hydroxide pellets were then added to obtain pH=12. The ethanol was vacuum evaporated at room temperature and the medium was then extracted with 3×200 ml of ethyl acetate. The organic phases were pooled, dried on Na₂SO₄ and vacuum concentrated: 15.8 g of the expected compound were obtained in the form of an orange oil (yield: 71%).

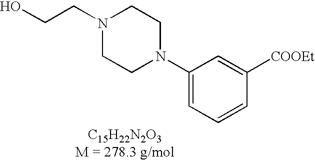

¹H NMR (CDCl₃) δ (ppm): 1.39 (t, J=7 Hz, 3H, —CH₃); 2.55-2.75 (m, 6H, CH₂CH₂O, CH₂N piperazine); 3.15-3.30 (m, 4H, CH₂N piperazine); 3.67 (t, J=5 Hz, 2H, CH₂O ); 4.37 (q, J=7 Hz, 2H, CH₂CO); 7.05-7.15 (m, 3H, Ar—H); 7.25-7.30 (m, 1H, Ar—H).

### EXAMPLE 70

### Ethyl 3-{4-[2-(2-ethoxy-phenylcarbamoyloxy)-ethyl]-piperazin-1-yl}-benzoate

9.23 ml (62.3 mmol) of 2-ethoxyphenylisocyanate were added to a solution of 15.7 g (56.5 mmol) of the compound of example 69 in 150 ml of dry acetonitrile. The mixture was refluxed for 14 h, then 0.9 ml (6.1 mmol) 2-ethoxyphenylisocyanate were added and the reaction mixutre was heated under reflux for another 6 h. After vacuum evaporation, the residue was taken up in 500 ml of ethyl ether and the insoluble material was filtered. The filtrate was vacuum concentrated and the residue loaded on a silica chromatography column (Cyclohexane 80/EtOAc 20+3% TEA) to produce 20.5 g of the expected compound in the form of a colorless oil (yield: 82%).

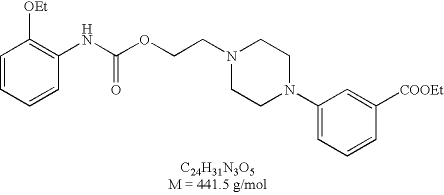

¹H NMR (CDCl₃) δ (ppm): 1.39 (t, J=7 Hz, 3H, —CH₃); 1.45 (t, J=7 Hz, 3H, —CH₃); 2.70-2.85 (m, 6H, CH₂CH₂O, CH₂N piperazine); 3.20-3.35 (m, 4H, CH₂N piperazine); 4.09 (q, J=7 Hz, 2H, PhOCH₂); 4.30-4.45 (m, 4H, CH₂OCON and PhCOOCH₂); 6.80-7.15 (m, 4H, Ar—H); 7.25-7.60 (m, 4H, Ar—H+NH); 8.05-8.15 (m, 1H, Ar—H).

HPLC/MS: RT=1.84 min/442 (M+H).

### EXAMPLE 71

### Sodium 3-{4-[2-(2-ethoxy-phenylcarbamoyloxy)-ethyl]-piperazin-1-yl}-benzoate

11.3 ml (11.3 mmol) of 1 N sodium carbonate were added to a solution of 5.0 g (11.3 mmol) of the compound of example 70 in 30 ml of ethanol. The mixture was heated for 60 h at 40° C. with stirring. After vacuum evaporation and drying, 4.5 g of the expected compound were obtained in the form of a white solid (yield: 92%).

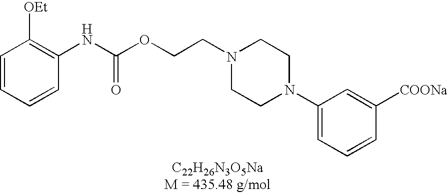

¹H NMR (DMDO-d6) δ (ppm): 1.35 (t, J=7 Hz, 3H, —CH₃); 2.50-2.70 (m, 6H, CH₂CH₂O, CH₂N piperazine); 3.05-3.15 (m, 4H, CH₂N piperazine); 4.05 (q, J=7 Hz, 2H, PhOCH₂); 4.22 (t, J=6 Hz, 2H, CHH₂OCON); 6.80-7.20 (m, 5H, Ar—H); 7.32 (d, J=8 Hz, 1H, Ar—H); 7.49 (s, 1H, Ar—H); 7.66 (d, J=8 Hz, 1H, Ar—H); 8.33 (s, 1H, NH).

HPLC/MS: RT=1.74 min/414 (M+H of the corresponding acid).

### EXAMPLES 72 TO 102

### Amide Derivatives of Example 71

To a suspension of PS-carbodiimide (Argonaut, 0.81 mmol/g, 74 mg, 0.060 mmol) in 0.33 ml of hydroxyazabenzotriazole solution (153 mM DCM {25% DMF}, 0.05 mmol), were added 0.5 ml of a solution of the compound of example 71 (66 mM DCM {50% DMF}, 0.033 mmol) and 0.5 ml of the amine solutions (60 mM DCM {20% DMF}, 0.030 mmol). After 16 h at room temperature, the reaction mixtures were treated overnight with PS-trisamine resin (Argonaut, 3.65 mmol/g, 65 mg, 0.24 mmol). The reaction mixtures were filtered and washed with 2×0.5 ml, vacuum concentrated and taken up in 0.5 ml of DCM, then treated overnight with PS-isocyanate resin (Argonaut, 1.44 mmol/g, 63 mg, 0.090 mmol). The compounds were obtained after concentrating the filtered solutions to dryness.

For example 72, N-tButyloxycarbonyl-ethylenediamine was the amine used, and the resulting compound was then hydrolyzed with 1 ml of DCM/TFA solution (1:1) for 2 h at room temperature. It was purified on cation exchange resin (SCX).

The compounds of examples 101 and 102 were also purified on cation exchange resin (SCX).

| | | | | | RT | | |
|---|---|---|---|---|---|---|---|
| n° | STRUCTURE | MW | Qty (mg) | Purity % | (min) | m | Ion |
| 72 | 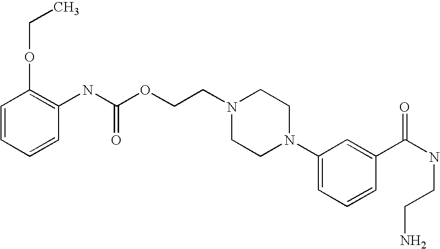 | 455.56 | 5.6 | 100 | 1.15 | 456.45 | M + 1 |
| 73 | 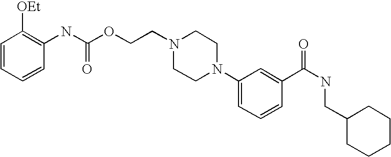 | 508.67 | 8.6 | 100 | 1.68 | 509.52 | M + 1 |
| 74 | 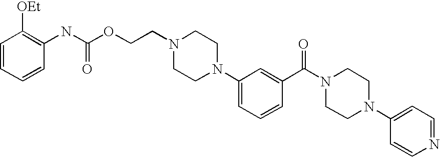 | 558.69 | 11.4 | 90.73 | 1.21 | 559.61 | M + 1 |
| 75 | 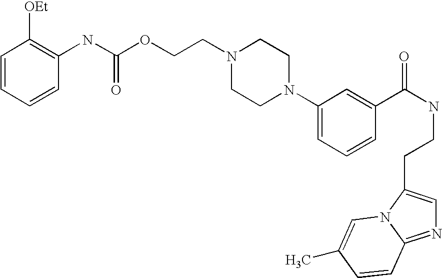 | 570.70 | 8.2 | 81.2 | 1.22 | 571.58 | M + 1 |
| 76 | 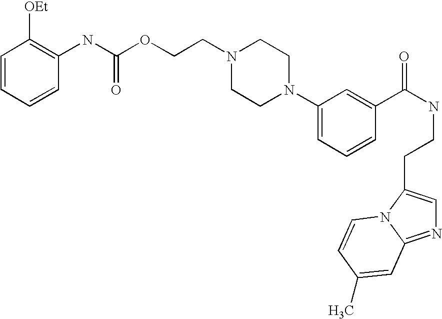 | 570.70 | 10.2 | 81.36 | 1.22 | 571.57 | M + 1 |
| 77 | 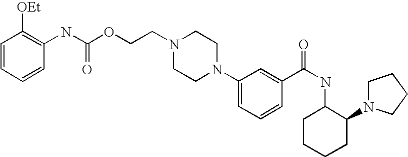 | 563.75 | 13 | 81.04 | 1.23 | 564.64 | M + 1 |
| 78 | 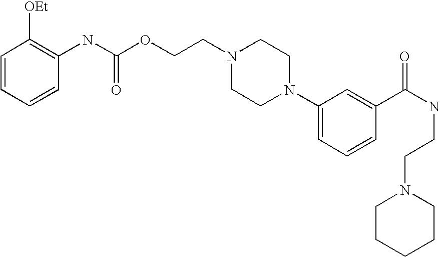 | 523.68 | 11.4 | 83.6 | 1.23 | 524.51 | M + 1 |
| 79 | 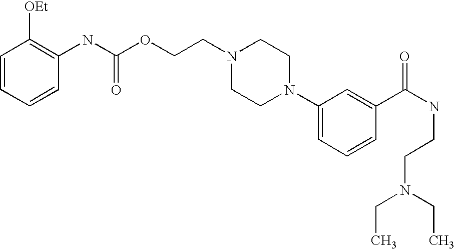 | 511.67 | 12.4 | 95.58 | 1.18 | 512.54 | M + 1 |
| 80 | 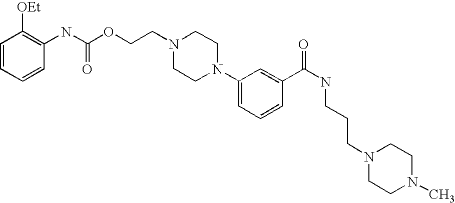 | 552.72 | 16.2 | 90 | 1.13 | 553.66 | M + 1 |
| 81 | 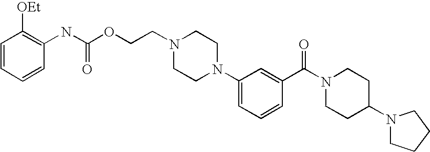 | 549.72 | 16.2 | 95.73 | 1.17 | 550.61 | M + 1 |
| 82 | 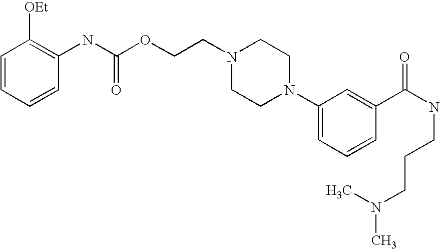 | 497.64 | 12.2 | 96.13 | 1.19 | 498.55 | M + 1 |
| 85 | 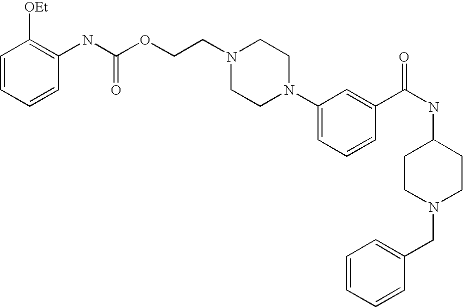 | 585.75 | 13.1 | 88.35 | 1.25 | 586.59 | M + 1 |
| 86 | 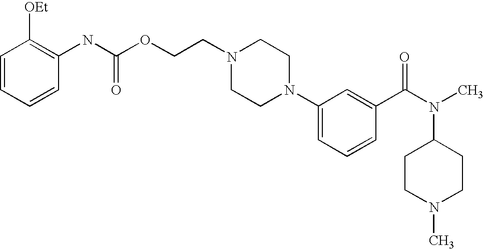 | 523.68 | 10 | 76.14 | 1.19 | 524.54 | M + 1 |
| 87 | 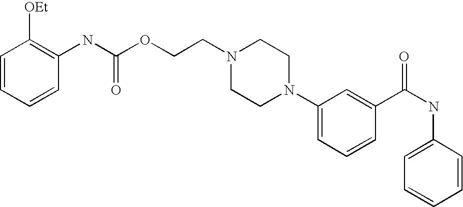 | 488.59 | 10.5 | 84.98 | 1.53 | 489.52 | M + 1 |
| 88 | 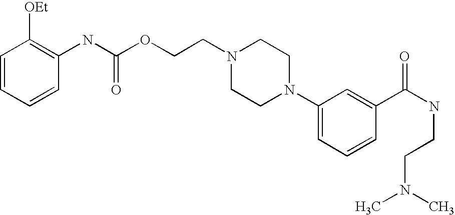 | 483.62 | 13.5 | 85.35 | 1.20 | 484.58 | M + 1 |
| 89 | 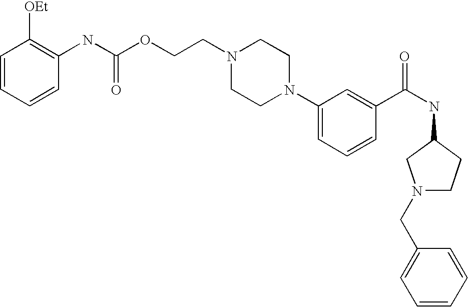 | 571.73 | 16.2 | 97.19 | 1.26 | 572.61 | M + 1 |
| 90 | 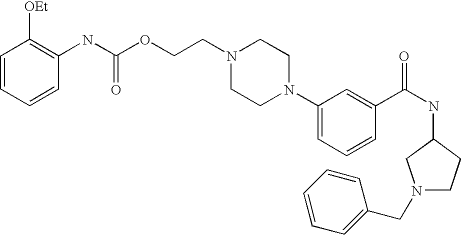 | 571.73 | 14.8 | 94.15 | 1.3 | 572.61 | M + 1 |
| 91 | 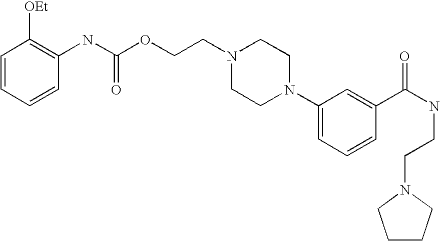 | 509.65 | 14.7 | 84.8 | 1.17 | 510.53 | M + 1 |
| 92 | 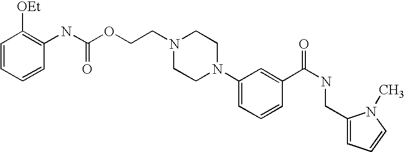 | 505.62 | 13.7 | 90.44 | 1.46 | 506.51 | M + 1 |
| 93 | 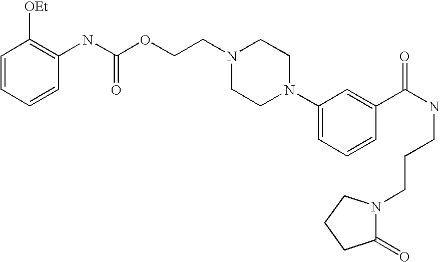 | 537.66 | 10.8 | 92.28 | 1.36 | 538.56 | M + 1 |
| 94 | 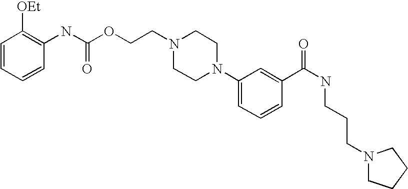 | 523.68 | 14.7 | 85.75 | 1.16 | 524.53 | M + 1 |
| 95 | 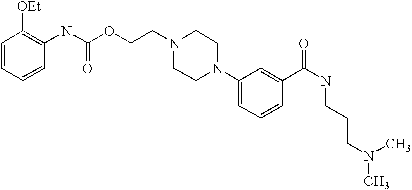 | 497.64 | 11.4 | 99 | 1.21 | 498.60 | M + 1 |
| 96 | 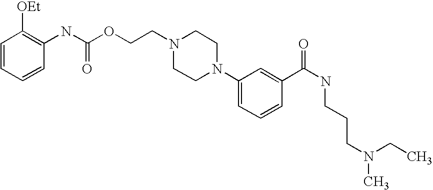 | 525.70 | 14.4 | 100 | 1.19 | 526.56 | M + 1 |
| 97 | 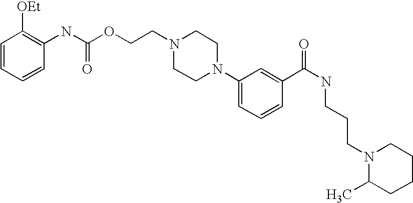 | 551.74 | 12.2 | 92.64 | 1.23 | 552.67 | M + 1 |
| 98 | 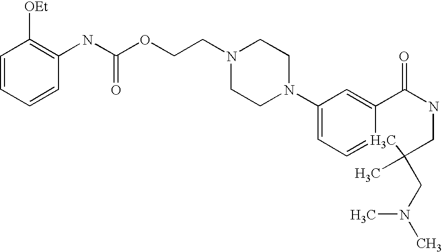 | 525.70 | 14.5 | 95.56 | 1.21 | 526.57 | M + 1 |
| 99 | 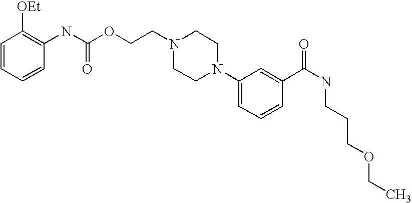 | 498.63 | 12.4 | 95.93 | 1.41 | 499.54 | M + 1 |
| 100 | 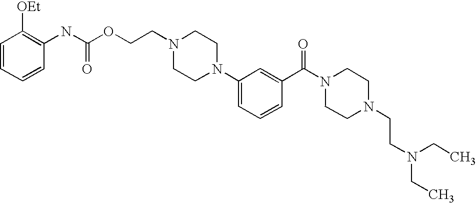 | 580.78 | 16.5 | 84.15 | 1.13 | 581.66 | M + 1 |
| 101 | 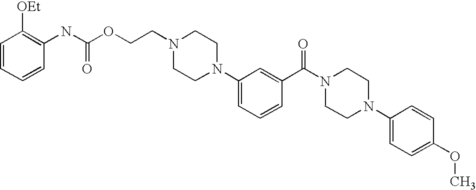 | 587.73 | 6.3 | 84.76 | 1.58 | 588.56 | M + 1 |
| 102 | 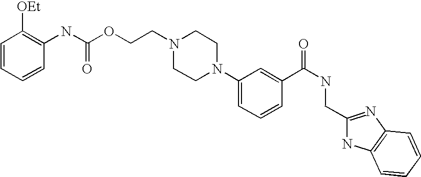 | 542.64 | 4.8 | 79.19 | 1.30 | 543.52 | M + 1 |

### EXAMPLE 103

### Methyl-(2-piperidin-1-yl-ethyl)amine

To a solution of 2 g (15.6 mmol) of 2-piperidin-1-yl-ethylamine in 20 ml of freshly distilled THF, at 0° C. in a nitrogen atmosphere, were added 2.60 ml (18.7 mmol) of triethylamine followed by dropwise addition of 1.79 ml (18.7 mmol) of ethyl chloroformate. The mixture was stirred for 16 h at room temperature. After evaporation to dryness, the residue was taken up in a mixture of ethyl acetate/saturated Na₂CO₃ solution. The aqueous phase was extracted two more times with ethyl acetate, then the pooled organic phases were dried on Na₂SO₄, filtered and vacuum evaporated. The reaction yielded 3.1 g of a colorless oil.

The crude product was taken up in 70 ml of freshly distilled THF. A suspension of 1.17 g (30.8 mmol) of mixed lithium and aluminium hydride was added dropwise to this solution cooled to 0° C., under nitrogen. The mixture was refluxed for 16 h and the reaction stopped by successivly adding 1.17 ml of water, 1.17 ml of 4 N sodium hydroxide, then 1.17 ml of water. After filtration and washing the precipitate with ether, the filtrate was evaporated to dryness and the residue was vacuum distilled (80° C., 5 mbar). The reaction produced 1.15 g of a colorless oil (yield: 52%).

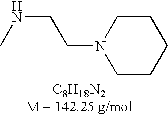

¹H NMR (CDCl₃) δ (ppm): 1.35-1.50 (m, 2H, CH₂—CH₂—CH₂); 1.50-1.75 (m, 4H, CH₂—CH₂—CH₂); 2.30-2.55 (m, 9H, 3CH₂N and NCH₃); 2.67 (t, J=6 Hz, 2H, CH₂N).

### EXAMPLES 104 TO 106

### Amide Derivatives of Example 71

0.128 ml (0.92 mmol) of triethylamine, 0.92 mmol of amine, 124 mg (0.92 mmol) of hydroxybenzotriazole, and 176 mg (0.92 mmol) of 1-(3-dimethylaminopropyl)-3-ethylcarbodiimide hydrochloride were successively added to a solution of 400 mg (0.92 mmol) of the compound of example 71 in 8 ml of dichloromethane. The mixture was stirred at room temperature for 24 h, then diluted with 10 ml of saturated Na₂CO₃ solution. The aqueous phase was extracted two more times with dichloromethane, then the pooled organic phases were dried on Na₂SO₄, filtered and vacuum evaporated. The residue was loaded on a silica chromatography column (eluent: EtOAc+3% triethylamine).

### EXAMPLE 104

### 2-(4-{3-[(2-piperidin-1-yl-ethyl)-methyl-carbamoyl]-phenyl}-piperazin-1-yl)-ethyl N-(2-ethoxy-phenyl)-carbamate

The reaction produced 0.210 g of a yellow oil (yield: 42%).

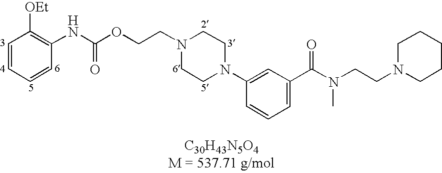

¹H NMR (CDCl₃) δ (ppm): 1.40-1.70 (m, 9H, CH₂—CH₂—CH₂and —CH₃); 2.20-2.35 (m, 2H, CH₂N); 2.40-2.70 (m, 4H, 2×CH₂N); 2.70-2.85 (m, 6H, CH₂CH₂O, CH₂N piperazine); 2.97 (s, 1.5H, NCH₃); 3.08 (s, 1.5H, NCH₃); 3.20-3.30 (m, 4H, CH₂N piperazine); 3.30-3.40 (m, 1H, CH₂NCH₃); 3.60-3.70 (m, 1H, CH₂NCH₃); 4.09 (q, J=7 Hz, 2H, PhOCH₂); 4.35 (t, J=6 Hz, 2H, CH₂OCON); 6.80-7.0 (m, 6H, Ar—H); 7.25-7.35 (m, 2H, Ar—H and NH); 8.05-8.15 (m, 1H, Ar—H).

HPLC: t=8.31 min

MS: 538 (MH⁺)

HPLC purity: 98%

### EXAMPLE 105

### 2-(4-{3-[(2-dimethylamino-ethyl)-methyl-carbamoyl]-phenyl}-piperazin-1-yl)-ethyl N-(2-ethoxy-phenyl)-carbamate

The reaction produced 0.150 g of a colorless oil (yield: 32%).

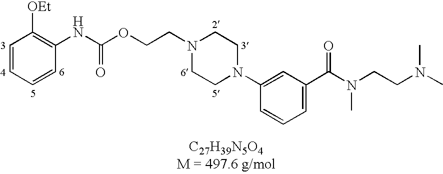

¹H NMR (CDCl₃) δ (ppm): 1.45 (t, J=7 Hz, 3H, —CH₃); 2.09 (s, 3H, NCH₃); 2.31 (s, 3H, NCH₃); 2.35-2.40 (m, 1H, CH₂N); 2.45-2.50 (m, 1H, CH₂N); 2.70-2.85 (m, 6H, CH₂CH₂O, CH₂N piperazine); 2.98 (s, 1.5H, CONCH₃); 3.08 (s, 1.5H, CONCH₃); 3.20-3.30 (m, 4H, CH₂N piperazine); 3.30-3.35 (m, 1H, CH₂NCH₃); 3.60-3.70 (m, 1H, CH₂NCH₃); 4.10 (q, J=7 Hz, 2H, PhOCH₂); 4.35 (t, J=6 Hz, 2H, CH₂OCON); 6.80-7.0 (m, 6H, Ar—H); 7.25-7.35 (m, 2H, Ar—H and NH); 8.05-8.15 (m, 1H, Ar—H).

HPLC: t=8.19 min

MS: 498 (MH⁺)

HPLC purity: 98%

### EXAMPLE 106

### 2-(4-{3-[(2-diethylamino-ethyl)-methyl-carbamoyl]-phenyl}-piperazin-1-yl)-ethyl N-(2-ethoxy-phenyl)-carbamate

The reaction produced 0.180 g of a colorless oil (yield: 38%).

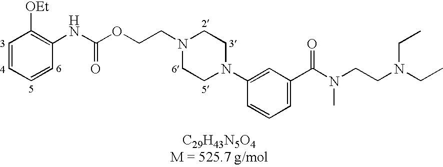

¹H NMR (CDCl₃) δ (ppm): 0.89 (t, J=7 Hz, 3H, NCH₂CH₃); 1.09 (t, J=7 Hz, 3H, NCH₂CH₃); 1.45 (t, J=7 Hz, 3H, OCH₂CH₃); 2.36 (q, J=7 Hz, 2H, NCH₂); 2.50-2.85 (m, 10H, 2×NCH₂, CH₂CH₂O, CH₂N piperazine); 3.00 (s, 1.5H, CONCH₃); 3.09 (s, 1.5H, CONCH₃); 3.20-3.30 (m, 4H, CH₂N piperazine); 3.35-3.40 (m, 1H, CH₂NCH₃); 3.55-3.65 (m, 1H, CH₂NCH₃); 4.09 (q, J=7 Hz, 2H, PhOCH₂); 4.35 (t, J=6 Hz, 2H, CH₂OCON); 6.80-7.0 (m, 6H, Ar—H); 7.25-7.35 (m, 2H, Ar—H and NH); 8.05-8.15 (m, 1H, Ar—H).

HPLC: t=9.00 min

MS: 526 (MH⁺)

HPLC purity: 99%

### EXAMPLE 107

### 2-[4-(3-nitrophenyl)-piperazin-1-yl]-ethanol

153 ml (1.170 mol, 5.5 eq) of 2-(piperazin-1-yl)ethanol were dissolved in 100 ml of DMSO, then a solution of 22.6 ml (0.214 mol) of 3-fluoro-nitrobenzene in 105 ml of DMSO was added. The mixture was heated at 100° C. for 30 h. After cooling to room temperature, the reaction medium was poured into 2 l of water and the precipitate was filtered. After vacuum drying, 41.8 g of the expected compound were obtained in the form of a yellow solid (yield: 78%).

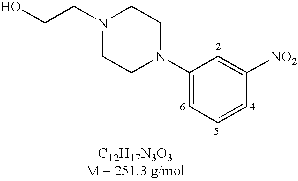

MP: 102-104° C.

¹H NMR (CDCl₃) δ (ppm): 2.60-2.80 (m, 6H, CH₂CH₂O, CH₂N piperazine); 3.25-3.35 (m, 4H, CH₂N piperazine); 3.69 (t, J=5 Hz, 2H, CH₂O); 7.17 (d, J=8 Hz, 1H, H6); 7.38 (t, J=8 Hz, 1H, H5); 7.67 (d, J=8 Hz, 1H, H4); 7.72 (s, 1H, H2).

HPLC/MS: RT=0.90 min/252 (M+H).

### EXAMPLE 108

### 1-[2-(tert-butyl-dimethyl-silanyloxy)-ethyl]4-(3-nitro-phenyl)-piperazine

7.05 ml (50.7 mmol, 3 eq) of triethylamine and 7.64 g (50.7 mmol, 3 eq) of tert-butyl-chloro-dimethylsilane were successively added to a suspension of 4.25 g (16.9 mmol) of the compound of example 107 in 150 ml of acetonitrile. The mixture was stirred at room temperature for 16 h, then refluxed for 2 h. After vacuum evaporation, the residue was taken up in a mixture of dichloromethane/saturated aqueous sodium carbonate solution. The organic phase was dried on Na₂SO₄, filtered and vacuum concentrated. The residue was loaded on a silica chromatography column (cyclohexane/EtOAc 80:20) to produce 5.03 g of the expected compound in the form of a yellow oil (yield: 81%).

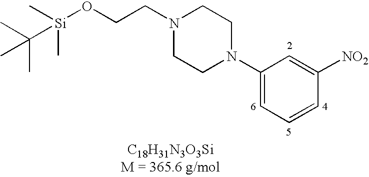

¹H NMR (CDCl₃) δ (ppm): 0.08 (s, 6H, 2×SiCH₃; 0.91 (s, 9H, C(CH₃)₃); 2.60 (t, J=6 Hz, 2H, CH₂CH₂O ); 2.65-2.75 (m, 4H, CH₂N piperazine); 3.20-3.30 (m, 4H, CH₂N piperazine); 3.80 (t, J=6 Hz, 2H, CH₂O); 7.14 (d, J=8 Hz, 1H, H6); 7.33 (t, J=8 Hz, 1H, H5); 7.66 (d, J=8 Hz, 1H, H4); 7.71 (s, 1H, H2).

HPLC/MS: RT=2.10 min/366 (M+H).

### EXAMPLE 109

### 1-[2-(tert-butyl-dimethyl-silanyloxy)-ethyl]4-(3-aminophenyl)-piperazine

5.03 g (13.8 mmol) of the compound of example 108 dissolved in 100 ml of methanol were vigorously stirred for 2 h in a hydrogen atmosphere (P=1 atm.), in the presence of 500 mg of palladium charcoal (10%). The catalyst was filtered and the residue vacuum concentrated to produce 4.37 g of the expected compound in the form of a colorless oil (yield: 94%).

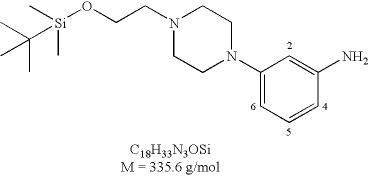

¹H NMR (CDCl₃) δ (ppm): 0.07 (s, 6H, 2×SiCH₃); 0.90 (s, 9H, C(CH₃)₃); 2.55 (t, J=6 Hz, 2H, CH₂CH₂O ); 2.60-2.70 (m, 4H, CH₂N piperazine); 3.10-3.20 (m, 4H, CH₂N piperazine); 3.60 (broad s, 2H, NH₂); 3.79 (t, J=6 Hz, 2H, CH₂O); 6.22 (d, J=8 Hz, 1H, H6); 6.25 (s, 1H, H2); 6.36 (d, J=8 Hz, 1H, H4); 7.04 (t, J=8 Hz, 1H, H5).

HPLC/MS: RT=1.83 min/336 (M+H).

### EXAMPLE 110

### N-(3-{4-[2-(tert-butyl-dimethyl-silanyloxy)-ethyl]-piperazin-1-yl}-phenyl)-3-diethylamino-propionanide

To a solution of 4.37 g (13.0 mmol) of the compound of example 109 in 130 ml of dichloromethane, were successively added 7.59 ml (54.6 mmol, 4.2 eq) of triethylamine, 2.36 g (13.0 mmol, 1 eq) of 3-(N-diethyl)aminopropionic acid hydrochloride, 1.93 g (14.3 mmol, 1.1 eq) of 1-hydroxybenzotriazole and 4.59 g (14.3 mmol, 1.1 eq) of O-(benzotriazol-1-yl)-N,N,N′,N′-tetramethyluronium tetrafluoroborate (TBTU). The mixture was stirred at room temperature for 72 h, then diluted with 100 ml of a saturated aqueous sodium carbonate solution. The organic phase was extracted two more times with saturated Na₂CO₃ solution, then dried on Na₂SO₄, filtered and vacuum concentrated. The residue was loaded on a silica chromatography column (dichloromethane 96/MeOH 4+0.4% NH₄OH then dichloromethane 90/MeOH 10+1% NH₄OH) to produce 2.87 g of the expected compound in the form of a colorless oil (yield: 47%).

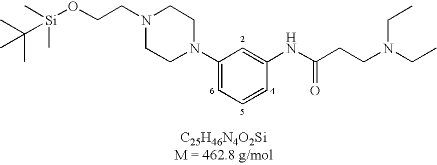

¹H NMR (CDCl₃) δ (ppm): 0.07 (s, 6H, 2×SiCH₂); 0.90 (s, 9H, C(CH₃)₃); 1.12 (t, J=7 Hz, 6H, 2×CH₃CH₂); 2.40-2.70 (m, 14H, CH₂CH₂O+COCH₂CH₂N+2×CH₃CH₂+2×CH₂N piperazine); 3.15-3.25 (m, 4H, 2×CH₂N piperazine); 3.80 (t, J=6 Hz, 2H, CH₂O); 6.63 (d, J=8 Hz, 1H, H6); 6.79 (d, J=8 Hz, 1H, H4); 7.15 (t, J=8 Hz, 1H, H5); 7.42 (s, 1H, H2).

HPLC/MS: RT=1.85 min/463 (M+H).

### EXAMPLE 111

### 3-diethylamino-N-{3-[4-(2-hydroxy-ethyl)-piperazin-1-yl]-phenyl}-propionamide

1.56 g (5.0 mmol, 2 eq) of tetrabutylammonium fluoride were added to a solution of 1.15 g (2.5 mmol) of the compound of example 110 in 10 ml of tetrahydrofuran. The mixture was stirred at room temperature for 12 h, then vacuum evaporated. The residue was taken up in 20 ml of dichloromethane and 20 ml of water. The aqueous phase was extracted two more times with 20 ml of dichloromethane. The organic phases were pooled, then dried on Na₂SO₄, filtered and vacuum concentrated. The residue was loaded on a silica chromatography column (dichloromethane 95/MeOH 5+0.5% NH₄OH) to produce 660 mg of the expected compound in the form of a colorless oil (yield: 76%).

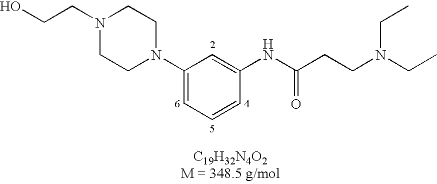

¹H NMR (CDCl₃) δ (ppm): 1.13 (t, J=7 Hz, 6H, 2×CH₃CH₂); 2.45-2.80 (m, 14H, CH₂CH₂O+COCH₂CH₂N+2×CH₃CH₂+2×CH₂N piperazine); 3.20-3.30 (m, 4H, 2×CH₂N piperazine); 3.66 (t, J=5 Hz, 2H, CH₂O ); 6.61 (d, J=8 Hz, 1H, H6); 6.79 (d, J=8 Hz, 1H, H4); 7.17 (t, J=8 Hz, 1H, H5); 7.45 (s, 1H, H2); 11.19 (broad s, 1H).

HPLC/MS: RT=0.29 min/349 (M+H).

### EXAMPLES 112 TO 119

### Phenylcarbamate Derivates of Example 111

0.8 ml of a solution of the compound of example 111 (0.188 M CH₃CN, 0.15 mmol) were added to 0.5 ml of a solution of substituted phenylisocyanate (0.3 M CH₃CN, 0.15 mmol). The reaction mixtures were stirred in an orbital shaker and maintained at 72° C. (external temperature) for 20 h. After returning to room temperature, 0.25 ml of substituted phenylisocyanate solution (0.3 M CH₃CN, 0.075 mmol) were added, and the reaction mixtures were stirred under the same conditions at 72° C. for 20 h. After vacuum evaporation, the residues were taken up in 2 ml of DMSO and purified by preparative HPLC (gradient: 10% B→100% B in 15 min). After vacuum evaporation, the expected products were taken up in a mixture of 2 ml of EtOAc and 2 ml of saturated Na₂CO₃ solution. The expected compounds were obtained after evaporating the organic phases to dryness.

The compounds were analyzed by HPLC/MS.

| No. | STRUCTURE | MW | RT (min) | m | Ion |
|---|---|---|---|---|---|
| 112 | 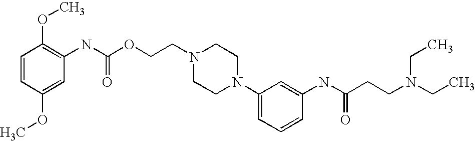 | 527.67 | 1.57 | 528.23 | M + 1 |
| 113 | 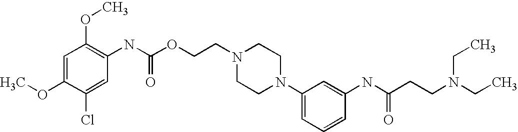 | 562.11 | 1.49 | 562.20 | M |
| 114 | 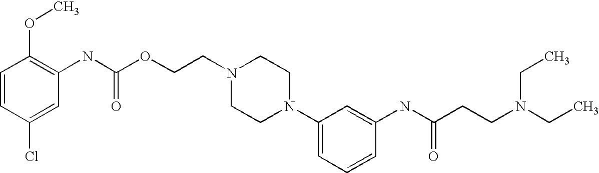 | 532.09 | 1.62 | 532.16 | M |
| 115 | 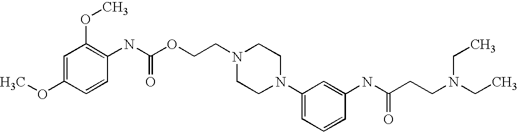 | 527.67 | 1.43 | 528.23 | M + 1 |
| 116 | 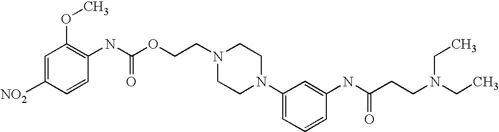 | 542.64 | 1.60 | 543.21 | M + 1 |
| 117 | 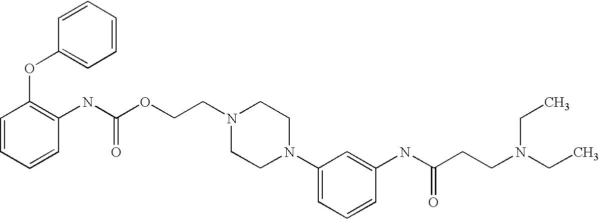 | 559.71 | 1.58 | 560.26 | M + 1 |
| 118 | 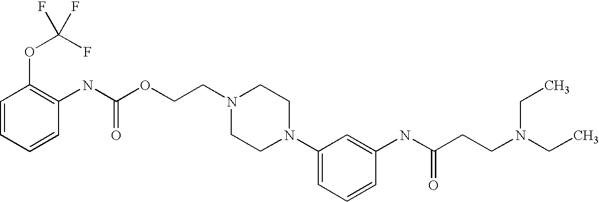 | 551.61 | 1.62 | 552.20 | M + 1 |
| 119 | 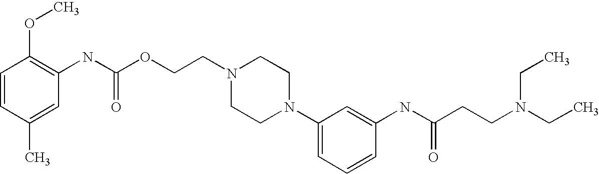 | 511.67 | 1.49 | 512.24 | M + 1 |

Biological Results:

The compounds of the invention were evaluated by measuring their affinity constant Ki determined by displacement of a radiolabelled ligand [³H]—GR113808 in rat glial cells stably expressing the human isoform h5-HT₄ₑ.

Confluent glial cells were washed twice with PBS and centrifuged at 300 g for 5 min. The pellet was used immediately and the cells were suspended in 10 volumes of HEPES (50 mM, pH 7.4, 4° C.) then homogenized in a teflon homogenizer and centrifuged at 40,000 g for 20 min. The pellet was again suspended in 15 volumes of HEPES. Binding displacement experiments were carried out in 500 ml of 50 mM HEPES buffer containing 20 μl of the radiolabelled ligand [³H]—GR113808 at a concentration of 0.2 nM for the isoform h5-HT₄ₑ or at a concentration equal to one-half the Kd of the radioligand for the other isoforms expressed in COS cells, 20 μl of competitor ligand at 7 different concentrations and 50 μl of membrane preparation (100-200 μg of protein determined by the Bradford method). Binding was carried out at 25° C. for 30 min and the reaction was stopped by rapid vacuum filtration (Brandel Harvester) on Whatman GF/B filters preincubated in 0.1% PEI to reduce nonspecific binding.

Membrane-bound radioactivity was retained on the filter, which was cut and washed with cold buffer (50 mM Tris-HCl, pH 7.4) and incubated overnight in 4 ml of scintillation fluid. Radioactivity was measured in a liquid scintillation counter (Beckmann LS 6500C). Binding data were obtained by computer-assisted linear regression. (Graph Prism Program, Graph Pad Software. Inc., San Diego, Calif.)

The results in the following table are given by way of example:

| Compound No. | Ki (nM) |
|---|---|
| 21 | 0.13 |
| 42 | 0.17 |
| 44 | 0.18 |
| 57 | 0.09 |
| 59 | 0.24 |
| 60 | 0.20 |
| 78 | 0.16 |
| 105 | 0.75 |

1-27. (canceled)

28. A compound represented by general formula (I): 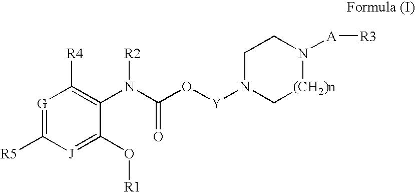
in which:
R₁ represents a lower alkyl, aryl, halogenoalkyl or lower arylalkyl group,
R₂ represents the hydrogen atom or a lower alkyl group,
A represents an aryl or heterocycle group, said group possibly being substituted by a substituent other than R3,
R₃ represents a group selected from among the following groups: 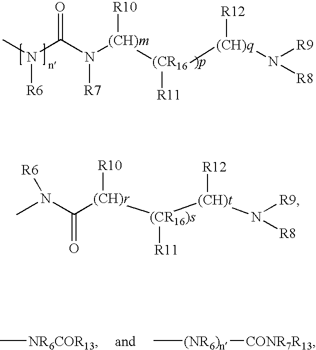
the groups R₇-R₁₂, which are the same or different, represent the hydrogen atom, an aryl group, a heteroaryl group, a heterocycle group, an arylalkyl group, a heteroarylalkyl group, a heterocycloalkyl group, a lower alkyl group, a cycloalkyl group, an alkoxyalkyl group, an alkylaminoalkyl group, an alkyl-COOR₁₇ group,
the groups R₇-R₁₂, taken two by two can additionally form, together with the linear chain supporting them, at least one ring saturated or not, such as in particular cycloalkyl, cycloalkylene, heterocycle,
the groups R₁₀-R₁₂ can also represent a COOR₁₇ group,
R₁₃ represents a lower alkyl group, a cycloalkyl group, an aryl group, a heterocycle, an arylalkyl group, a heteroarylalkyl group, a heterocycloalkyl group, a cycloalkylcarboxy group, an alkyl-COOR₁₇ group, an alkoxyalkyl group, an imidazopyridinylalkyl group, a trifluoroalkyl group or a heteroarylthioalkyl group, it being understood that R₁₃ cannot represent the methyl group or the ethyl group, in the case where A represents a phenyl, R₂ represents the hydrogen atom, G and J represent the CH group, R₃ represents NR₆COR₁₃ or —(NR₆)_{n′}—CONR₇R₁₃ where R₆ and/or R₇ represent the hydrogen atom,
n is 1 or 2; n′ is 0 or 1, m, p, q, r, s and t are integers comprised between 0 and 2 inclusive, r, s and t not simultaneously being 0,
Y represents a linear or branched alkylene chain, having 2 to 5 carbon atoms,
J represents a C—R₁₄ group or the nitrogen atom
G represents a C—R₁₅ group or the nitrogen atom
R₆, R₁₆ and R₁₇, which are the same or different, represent the hydrogen atom or a lower alkyl group,
R₄, R₅, R₁₄ and R₁₅ taken individually represent the hydrogen atom, a halogen atom, a lower alkyl group, an alkoxy, alkylthio, alkylsulfonyl, alkylsulfoxide, trifluoromethyl, nitro, cyano, carboxy, alkylcarboxy, alkylamino or dialkylamino group,
or, when G or J are not the nitrogen atom, the groups OR₁ and R₁₄ and/or the groups R₁₄ and R₅ and/or the groups R₁₅ and R₅ and/or the groups R₁₅ and R₄ can form, together with the aromatic ring to which they are attached, a ring saturated or not,
said alkyl, cycloalkyl, aryl, arylalkyl, heteroaryl, heterocycle, heterocycloalkyl, heteroarylalkyl, alkylaminoalkyl, alkoxy, alkoxyalkyl, alkylthio and alkylcarboxy groups,
and said ring, being substituted or not,
and their salts, optical and geometrical isomers or their mixtures.

29. The compound represented by general formula (I) according to claim 28, in which R1 represents a lower alkyl group and preferably a methyl or ethyl group.

30. The compound represented by general formula (I) according to claim 28, in which:
A represents a phenyl, a pyrimidine, a pyridazine or a pyrazine and/or
n=1 and/or
n′=1 and/or
Y is an alkylene chain having 2 or 3 carbon atoms, preferably linear, and/or
R₂ is a hydrogen atom, and/or
R₃ represents a group selected from among the following: 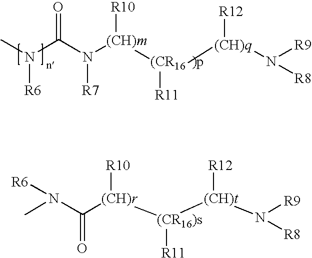
R₄ is a hydrogen atom, and/or
R₆ is a hydrogen atom, and/or
G is a CH group, and/or
J is a CH group.

31. The compound represented by general formula (I) according to claim 28, in which:
A represents a phenyl, a pyrimidine, a pyridazine or a pyrazine and/or
n=1 and/or
n′=0 and/or
Y is an alkylene chain having 2 or 3 carbon atoms, preferably linear, and/or
R₂ is a hydrogen atom, and/or
R₃ represents a group selected from among the following: 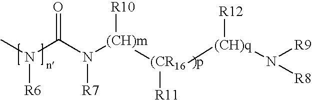
R₄ is a hydrogen atom, and/or
G is a CH group, and/or
J is a CH group.

32. The compound represented by general formula (I) according to claim 28, in which:
A represents a phenyl, a pyrimidine, a pyridazine or a pyrazine and/or
n=1 and/or
Y is an alkylene chain having 2 or 3 carbon atoms, preferably linear, and/or
R₂ is a hydrogen atom, and/or
R₄ is a hydrogen atom, and/or
R₅ is a hydrogen atom, and/or
G is a CH group, and/or
J is a CH group, and/or
R₃ represents a group selected from among the following: 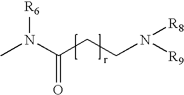
where R₆ is a hydrogen atom or a lower alkyl group (in particular methyl) and r represents 0, 1 or 2 (in particular 1 or 2).

33. The compound represented by general formula (I) according to claim 28, in which:
A represents a phenyl, a pyrimidine, a pyridazine or a pyrazine and/or
n=1, and/or
Y is an alkylene chain having 2 or 3 carbon atoms, preferably linear, and/or
R₂ is a hydrogen atom, and/or
R₄ is a hydrogen atom, and/or
R₅ is a hydrogen atom, and/or
G is a CH group, and/or
J is a CH group, and/or
R₃ represents a group selected from among the following: 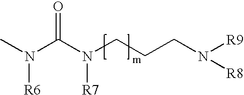
where R₆ is a hydrogen atom or a lower alkyl group (in particular methyl), R₇ is a hydrogen atom or a lower alkyl group (in particular methyl), and m is an integer comprised between 0 and 2 inclusive (in particular 0 or 1).

34. The compound represented by general formula (I) according to claim 28, in which:
A represents a phenyl, a pyrimidine, a pyridazine or a pyrazine and/or
n=1, and/or
Y is an alkylene chain having 2 or 3 carbon atoms, preferably linear, and/or
R₂ is a hydrogen atom, and/or
R₄ is a hydrogen atom, and/or
R₅ is a hydrogen atom, and/or
G is a CH group, and/or
J is a CH group, and/or
R₃ represents a group selected from among the following: 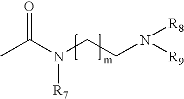
where R₇ is a hydrogen atom or a lower alkyl group (in particular methyl) and m represents 1 or 2.

35. The compound represented by general formula I according to claim 28, in which R3 represents a —NR₆—COR₁₃ or —(NR₆)_{n′}—CONR₇R₁₃ group, with R₁₃ representing a cycloalkyl group, a heterocycle, an arylalkyl group, a heteroarylalkyl group, a heterocycloalkyl group, an alkylcarboxy group, a cycloalkylcarboxy group, an alkyl-COOR₁₇ group, an imidazopyridinylalkyl group, a trifluoroalkyl group or a heteroarylthioalkyl group.

36. The compound represented by general formula I according to claim 28, in which R3 represents a —CONR₇R₁₃ group, with R₁₃ representing a cycloalkyl group, a heterocycle, an arylalkyl group, a heteroarylalkyl group, a heterocycloalkyl group, an alkylcarboxy group, a cycloalkylcarboxy group, an alkyl-COOR₁₇ group, an alkoxyalkyl group, an imidazopyridinylalkyl group, a trifluoroalkyl group or a heteroarylthioalkyl group.

37. The compound represented by formula (I) according to claim 28, in which A represents a phenyl, possibly substituted.

38. The compound represented by formula (I) according to claim 28, in which Y is an alkylene chain containing 2 or 3 carbons.

39. The compound according to claim 28, selected in the group consisting of compounds of examples Nos. 9 to 46, and salts thereof.

40. The compound according to claim 28, selected in the group consisting of compounds of examples Nos. 47 to 67, and salts thereof.

41. The compound according to claim 28, selected in the group consisting of compounds of examples Nos. 72 to 102 and 104 to 106, and salts thereof.

42. The compound according to claim 28, selected in the group consisting of compounds of examples Nos. 112 to 119, and salts thereof.

43. The compound according to claim 28, selected in the group consisting of the following:
2-(4-{3-[3-(1-ethyl-pyrrolidin-2-ylmethyl)-ureido]-phenyl}-piperazin-1-yl)-ethyl-N-(2-ethoxy-phenyl)carbamate,
2-(4-{3-[(1-methyl-1,2,5,6-tetrahydro-pyridine-3-carbonyl)-amino]-phenyl}-piperazin-1-yl)-ethyl-N-(2-ethoxy-phenyl)carbamate,
2-{4-[3-(3-amino-propionylamino)-phenyl]-piperazin-1-yl}-ethyl ester-N-(2-ethoxy-phenyl)carbamate,
2-(4-{3-[2-amino-3-(4-hydroxy-phenyl)-propionylamino]-phenyl}-piperazin-1-yl)-ethyl-N-(2-ethoxy-phenyl)carbamate,
2-[4-(3-{3-[3-(4-methyl-piperazin-1-yl)-propyl]-ureido}-phenyl)-piperazin-1-yl]-ethyl-N-(2-ethoxy-phenyl)carbamate,
2-(4-{3-[(4-pyrrolidin-1-yl-piperidine-1-carbonyl)-amino]-phenyl}-piperazin-1-yl)-ethyl N-(2-ethoxy-phenyl)carbamate,
2-(4-{3-[2-piperidin-1-yl-ethylcarbamoyl]-phenyl}-piperazin-1-yl)-ethyl-N-(2-ethoxy-phenyl)carbamate,
2-(4-{3-[(2-dimethylamino-ethyl)-methyl-carbamoyl]-phenyl}-piperazin-1-yl)-ethyl-N-(2-ethoxy-phenyl)carbamate, and the salts thereof.

44. Intermediate compounds useful for preparing products according to claim 28 which are ethyl 3-{4-[2-(2-ethoxy-phenylcarbamoyloxy)-ethyl]-piperazin-1-yl}-benzoate, sodium 3-{4-[2-(2-ethoxy-phenylcarbamoyloxy)-ethyl]-piperazin-1-yl}-benzoate or one of the addition salts of same.

45. A pharmaceutical composition comprising at least one compound according to claim 28.

46. The pharmaceutical composition according to claim 45, for the treatment or prophylaxis of diseases involving the 5-HT4 receptor.

47. The pharmaceutical composition according to claim 45, for the treatment or prophylaxis of gastrointestinal disorders, central nervous system disorders, cardiac diseases, urological diseases, pain or migraine.

48. A method of treatment or prophylaxis of the human or animal body by administering, into a human or animal body in need of such treatment, an effective amount of at least one compound according to claim 28.

49. A method for treating gastrointestinal disorders, central nervous system disorders, cardiac diseases, urological diseases, pain or migraine, by administering into a mammal, more particularly a human, in need of such treatment an effective amount of at least one compound according to claim 28.

50. A method for preparing a compound according to claim 28, wherein a product represented by formula (II) is reacted with a product represented by formula (III): 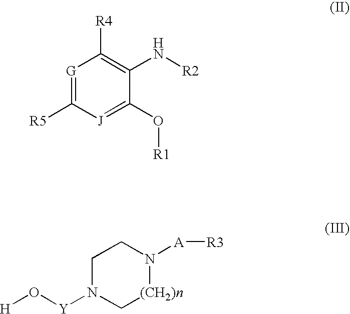
wherein the groups R1, R2, R3, R4, R5, A, Y, J, G and n are defined as in claim 28, in the presence of a carbonyl donor reagent, preferably triphosgene, and the resulting product is recovered.

51. A method for preparing a compound according to claim 28, wherein a product represented by formula (IV) is reacted with a product represented by formula (III): 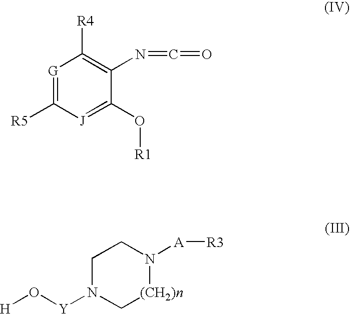
in which the groups R1, R3, R4, R5, A, Y, J, G and n are defined as in claim 28, in an aprotic solvent, preferably tetrahydrofuran.

52. A method for preparing a compound according to claim 28, wherein a product represented by formula (V) is reacted with a product represented by formula (VI) or (VII): 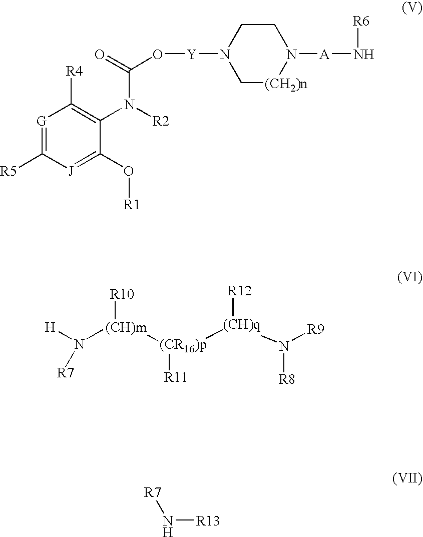
in which the groups R1-R13, R16, A, Y, J, G and n, m, p, q are defined as in claim 28, in an aprotic solvent, preferably tetrahydrofuran, in the presence of a carbonyl donor reagent, preferably triphosgene.

53. A method for preparing a compound according to claim 28, wherein a product represented by formula (V) is reacted with a product represented by formula (VIII) or (IX): 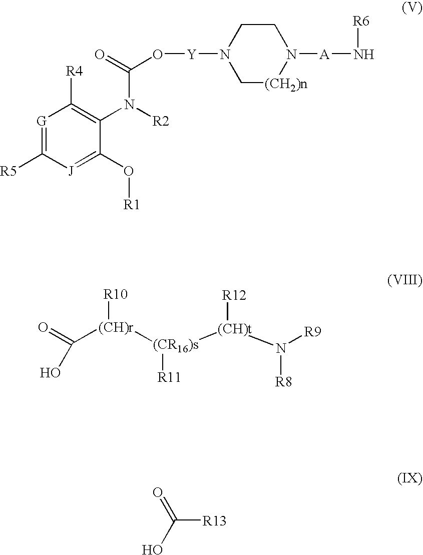
in which the groups R1-R6, R8-R13, R16, A, Y, J, G and n, r, s and t are defined as in claim 28, in an aprotic solvent preferably tetrahydrofuran, in the presence of a classical coupling agent, preferably DCC on a solid support or EDCI.

54. A method for preparing a compound according to claim 28, wherein a product represented by formula (X) is reacted with a product represented by formula (VI) or (VII): 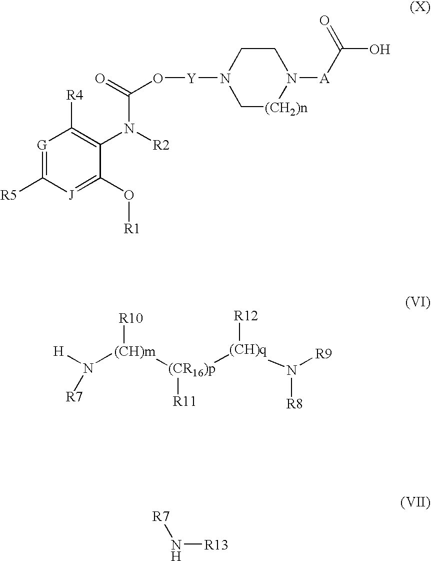
in which the groups R1-R5, R7-R13, R16, A, Y, J, G and n, m, p and q are defined as in claim 28, in an aprotic solvent preferably tetrahydrofuran, in the presence of a classical coupling agent, preferably DCC on a solid support or EDCI.
